(19) United States
(12) Patent Application Publication
(10) Pub. No.: US 2006/0258729 A1
(43) Pub. Date: Nov. 16, 2006

(54) Antibacterial pyrazole carboxylic acid hydrazides

(76) Inventor: Michelle M. Butler, Auburn, MA (US)

Correspondence Address: Leon R Yankwich;Yankwich & Associates, 201 Broadway, Cambridge, MA, 02139, US

(21) Appl. No.: 10/527,037

(22) PCT Filed: Sep. 10, 2003
(86) PCT No.: PCT/US03/28392

§ 371 Date: Mar. 8, 2005

Related U.S. Application Data
(60) Provisional application No. 60/409,439, filed on Sep. 10, 2002.

(51) Int. Cl. A61K 31/416; A61K 31/4152

(52) U.S. Cl. 514/406

(57) ABSTRACT

Methods and compositions comprising pyrazole carboxylic acid hydrazide compounds are described that are useful for treating bacterial infections, inhibiting bacterial growth, and inhibiting the activity of bacterial DNA polymerase III.

### CROSS-REFERENCE TO RELATED APPLICATIONS

This application claims priority to U.S. Provisional Application No. 60/409,439, filed Sep. 10, 2002.

### STATEMENT REGARDING FEDERALLY SPONSORED RESEARCH

This invention was made with support from the United States Government under SBIR Grant 1 R43 AI49595-01A1 awarded by the National Institutes of Health, National Institute of Allergy and Infectious Disease. The Government has certain rights in the invention.

### GENERAL FIELD OF THE INVENTION

This invention relates generally to antimicrobial compounds. In particular, the invention describes pyrazole carboxylic acid hydrazide compounds and their use as antibacterial agents.

### BACKGROUND OF THE INVENTION

Bacterial pathogens, and especially, Gram-positive pathogens pose a continuing and serious threat to public health. Two such pathogens, Staphylococcus aureus and Enterococcus fecalis/fecium, are primarily nosocomial (hospital-acquired) pathogens; together, they presently account for the majority of nosocomial infections. A third organism, Streptococcus pneumoniae, is a community-acquired pathogen that also causes serious bacterial infections.

Staphylococcus aureus is currently the most frequent cause of nosocomial bacteremia and skin/wound infection and the second most frequent cause of nosocomial lower respiratory infection. Enterococcus fecalis/fecium ranks third behind Staphylococcus aureus and the Gram-negative Escherichia coli as a cause of nosocomial septicemia, endocarditis, and infections of wounds and the urinary tract. Streptococcus pneumoniae causes several serious and potentially life-threatening diseases. In the United States it is estimated that Streptococcus pneumoniae accounts annually for 6,000 cases of pneumococcal meningitis, a half million cases of pneumonia, 55,000 cases of bacteremia, and 6 million cases of otitis media. Annual mortality from Streptococcus pneumoniae-induced disease is estimated to be 40,000 in the United States and 3-5 million globally.

There is a rapidly growing global crisis in the clinical management of life-threatening infectious disease caused by multi-antibiotic-resistant strains of a variety of Gram-negative and Gram-positive bacterial pathogens, including various strains and species of the Gram-positive pathogens Streptococcus, Enterococcus, and Staphylococcus, and various strains and species of pathogenic Gram-negative bacteria such as Escherichia, Salmonella, Pseudomonas, Helicobacter, Legionella, Shigella, Yersinia and Neisseria. New antibacterial compounds that are effective against such important bacterial pathogens must be developed as part of the effort to successfully meet this worldwide public health crisis.

### SUMMARY OF THE INVENTION

The present invention is based on the discovery that pyrazole carboxylic acid hydrazide compounds have useful antibacterial activities owing to the ability to inhibit DNA polymerase III, which is essential for proper replication of the bacterial chromosome. A DNA polymerase III enzyme is either one of two classes, i.e., a DNA polymerase IIIC (pol IIIC) or a DNA polymerase IIIE (pol IIIE), and a bacterial cell may possess a species of one or both of these classes. Thus, Gram-positive and mycoplasma bacteria possess both pol IIIC and pol IIIE classes of DNA polymerase III, and Gram-negative bacteria possess the pol IIIE class of DNA polymerase III. The pyrazole carboxylic acid hydrazide compounds described herein are capable of inhibiting DNA polymerase III activity and may be used as antibiotics to inhibit the growth of a variety of bacteria, including various species or strains of Gram-positive bacteria, Gram-negative bacteria, and mycoplasma bacteria. These antibacterial compounds are the first “non-nucleotide” inhibitors of a DNA polymerase, i.e., the pyrazole carboxylic acid hydrazide compounds described herein do not possess any of the heterocyclic nitrogenous bases (guanine, adenine, cytosine, thymine, uracil) commonly found in deoxyribonucleic acid (DNA) or ribonucleic acid (RNA). These compounds may be administered to prevent or to treat bacterial infections in animals, including humans and other mammals, and may also be used in various non-clinical applications, including research and manufacturing, such as to prevent bacterial contamination in eukaryotic cell cultures, or in biochemical studies, as novel inhibitors of one or more species of DNA polymerase III.

The invention features methods and compositions comprising a pyrazole carboxylic acid hydrazide compound of formulas 1a and 1b shown below: 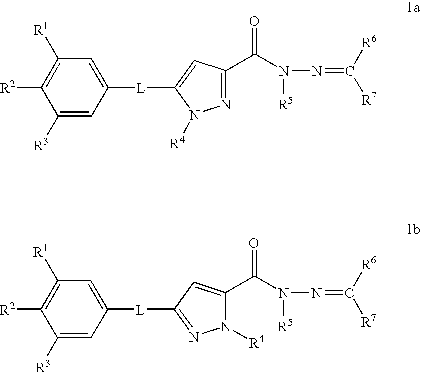
wherein R¹, R², and R³ are each, independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;

R⁴ and R⁵ are, independently selected from the group consisting of H, substituted or unsubstituted alkyl, substituted or unsubstituted alkenyl, substituted or unsubstituted alkynyl, and substituted or unsubstituted cycloalkyl wherein it is understood that, when R⁴ is H, two tautomeric forms depicted by structures 1a and 1b may exist;

R⁶ and R⁷ are, independently, H, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted alkylaryl, substituted or unsubstituted heteroaryl, or R⁶ and R⁷ are atoms that form part of a aromatic or non-aromatic, heterocylic or carbocyclic ring or ring system, comprising either a monocyclic ring or a fused ring system, having ring atoms selected from the group consisting of substituted or unsubstituted carbon, nitrogen or sulfur; and

L may be absent or selected from the group consisting of linkers having 1-6 atoms in contiguous linear connectivity; or

an enantiomeric or diastereomeric form or a pharmaceutically acceptable salt thereof.

This invention also provides pharmaceutical compositions comprising a pyrazole carboxylic acid hydrazide compound, methods for inhibiting the growth of bacteria using a pyrazole carboxylic acid hydrazide compound, and methods for therapeutically or prophylactically (i.e., preventative therapy) treating an animal, including a human, which has, is suspected of having, or is susceptible to a bacterial infection comprising administering to the individual a pyrazole carboxylic acid hydrazide compound described herein.

Particularly preferred are methods and compostions for treating or preventing infections, inhibiting the activity of DNA polymerase III, and inhibiting growth of Gram-positive bacteria, mycoplasma bacteria, and/or Gram-negative bacteria.

The details of one or more embodiments of the invention are set forth in the accompanying description below. Other features, objects, embodiments, and advantages of the invention will be apparent from the description and from the claims.

### BRIEF DESCRIPTION OF THE DRAWINGS

FIG. 1A shows a Lineweaver-Burk double reciprocal plot of data for the inhibition of B. subtilis pol IIIC by Compound 17, indicative of an uncompetitive inhibition mechanism.

FIG. 1B shows a Lineweaver-Burk double reciprocal plot of data for the inhibition of B. subtilis pol IIIC by HB-EMAU, indicative of a competitive inhibition mechanism.

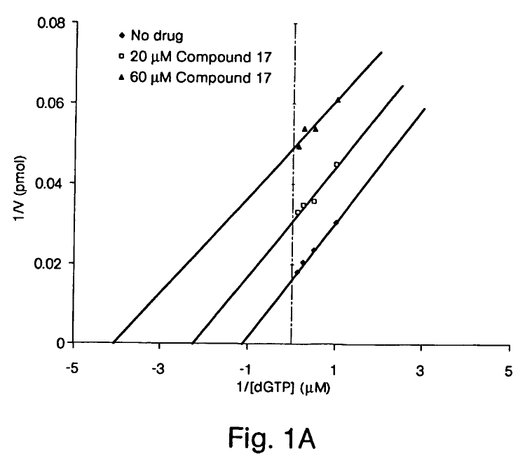

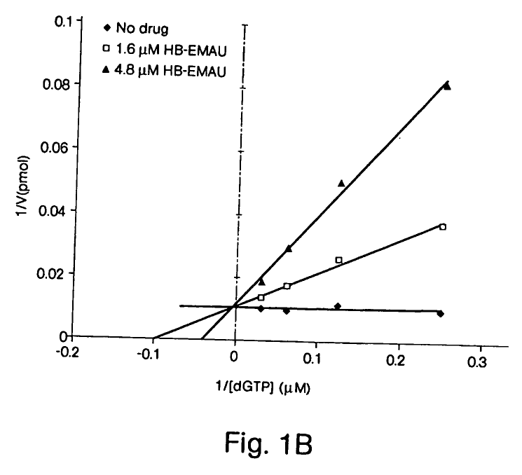

The variable substrate in the experiments for FIGS. 1A and 1B was dGTP. See text for details.

### DETAILED DESCRIPTION OF THE INVENTION

The invention is based on the discovery that pyrazole carboxylic acid hydrazide compounds have the ability (biochemical property) to inhibit the activity of one or more species of the bacterial enzyme DNA polymerase III involved in replication of the bacterial chromosome. Structurally, the pyrazole carboxylic acid hydrazide compounds described herein are unlike any other currently available antibacterial compound. With respect to inhibition of DNA polymerase III activity, the pyrazole carboxylic acid hydrazide compounds described herein also appear to be unlike any classically described DNA polymerase III inhibitor. Accordingly, the invention features methods and compositions comprising a pyrazole carboxylic acid hydrazide compound described herein for treating or preventing bacterial infections in an individual; for inhibiting growth of strains or species of Gram-positive bacteria, mycoplasma bacteria, and/or Gram-negative bacteria; and for inhibiting the activity of DNA polymerase III.

In order that the invention may be more fully understood, the following definitions apply:

The term “acyl” means the radical —C(O)R, wherein R is selected from alkyl, aryl, alkylaryl, arylakyl (such as benzyl), alkylarylalkyl, heterocyclyl, heterocyclylalkyl, carbocyclyl, carbocyclylalkyl, alkoxyalkyl (such as methoxymethyl), alkoxyalkyl, aryloxyalkyl (such as phenoxymethyl), poly(alkyloxy)alkyl (such as polyethers like poly(methoxy)methyl), aryl (such as phenyl optionally substituted with halo, lower alkyl or lower alkoxy), arylalkyl, and alkylaryl. Specific examples of acyl segments include, without limitation, acetyl, propionyl, butyryl, pentanoyl, 3-methylbutyryl, hydrogen succinyl, 3-chlorobenzoyl, benzoyl, pivalyl, mesyl, propionyl, valeryl, caproic, capryl, lauryl, myristyl, palmityl, stearyl and oleyl.

The term “alkyl” means a saturated straight chain or branched, primary, secondary, or tertiary hydrocarbon radical, typically C₁-C₁₈ (for example, C₁-C₁₀, such as C₁-C₆) including, without limitation, methyl, ethyl, propyl, butyl, pentyl, hexyl, isopropyl, isobutyl, sec-butyl, t-butyl, isopentyl, amyl, and t-pentyl. For the purposes of this invention, any carbon in the alkyl segment may be substituted with oxygen (O), sulfur (S), or nitrogen (N). Further, alkyl segments may optionally be substituted with one or more conventionally used alkyl substituents, such as amino, alkylamino, alkoxy, alkylthio, oxo, halo, acyl, nitro, hydroxyl, cyano, aryl, alkylaryl, aryloxy, arylthio, arylamino, carbocyclyl, carbocyclyloxy, carbocyclylthio, carbocyclylamino, heterocyclyl, heterocyclyloxy, heterocyclylamino, heterocyclylthio, and the like. Unless otherwise specified, when the term “alkyl” is used together in a compound term (such as “carbocyclylalkyl” or “arylalkyl”), the number of carbon atoms or ring numbers used in connection with such compound term shall not include the atoms of the alkyl portion of the moiety (unless the other portion of the moiety does not contain any carbon atoms). In such cases, the alkyl portion will typically have the chain lengths set forth in the definition above for other alkyl moieties.

The term “alkylamino” means an amino segment substituted with one or two alkyl groups (i.e., includes dialkyl amino radicals) wherein the alkyl groups may be the same or different.

The term “alkylaryl” means an aryl radical substituted with one or more alkyl substituents.

The term “alkenyl” means an alkyl radical having one or more double bonds. Alkenyl groups containing three or more carbon atoms may be straight or branched.

The term “alkynyl” means an alkyl radical having one or more triple bonds. Alkynyl groups containing three or more carbon atoms may be straight or branched.

The term “amino” means a —NH₂, —NHR⁸, or —NR⁹R¹⁰, wherein R⁸, R⁹, and R¹⁰ may be the same or different, and independently represent a conventionally used amino substituent. Preferably, R⁸, R⁹, and R¹⁰ are independently selected from the group consisting of optionally substituted alkyl (such as lower alkyl), aryl, and alkylarylalkyl.

The term “antibacterial activity” (and equivalent terms used herein) of a compound or composition of the invention means either having a measurable minimum inhibitory concentration (MIC) value in vitro against the growth of whole, intact bacteria, or producing a clinically recognizable improvement of one or more symptoms of a bacterial infection in vivo in a patient in need thereof. MIC may be measured by techniques known to those skilled in the art, for example, testing a compound for anti-microbial activity against one or more species or strains of bacteria on a solid medium or in a liquid medium supplemented with varying concentrations of the test compound. The compounds described herein may have an antibacterial activity against one or more strains or species of Gram-positive bacteria, Gram-negative bacteria, and/or mycoplasma bacteria. Of particular interest with respect to inhibiting growth of Gram-positive bacteria are compounds described herein that have an antibacterial activity against one or more strains or species of pathogenic or potentially pathogenic Gram-positive bacteria, such as those selected from the group consisting of Streptococcus, Enterococcus, Staphylococcus, Bacillus, Clostridium, and Listeria. Of particular interest with respect to inhibiting growth of Gram-negative bacteria are compounds described herein that have an antibacterial activity against one or more strains or species of such clinically relevant Gram-negative bacteria, such as those selected from the group consisting of Escherichia, Salmonella, Pseudomonas, Helicobacter, Legionella, Shigella, Yersinia and Neisseria. A clinically recognizable improvement of a symptom of a bacterial infection is any medically-recognized improvement in the health of a patient, including, but not limited to, survival or recovery of the patient from the bacterial infection, reduction in fever, tissue or wound healing, decrease in pain, increase in patient physical or mental vigor, increase in patient appetite, restoration of normal heartbeat, restoration of normal breathing, restoration of normal levels of white blood cells in blood, decrease in titer of antibodies to bacterial antigens in blood or other tissues, and reduction in titer of pathogenic bacteria in biological samples obtained from the patient. Antibacterial activity also encompasses the desirable prevention (prophylactic treatment) of the manifestation or presentation of one or more symptoms of bacterial infection in an individual at risk for bacterial infection.

The terms “antibiotic” and “antimicrobial” have the meaning normally employed in the art, including any compound that is effective at inhibiting the growth of or destroying microorganisms, particularly wherein such microorganisms are pathogenic or potentially pathogenic bacteria. An antibiotic may be produced from living cells or synthesized chemically in whole or in part. Accordingly, owing to their antibacterial activity, pyrazole carboxylic acid hydrazide compounds described herein are also referred to as antibiotics or antimicrobials.

The term “aryl” means a 5-8 membered monocyclic aromatic ring or a polycyclic aromatic ring or ring system having 5-8 ring members in each ring thereof, which may be carbocyclic or heterocyclic and may be unsubstituted or substituted with one or more substituents including (but not limited to) alkyl (for example, lower alkyl), hydroxy, alkoxy (for example, lower alkoxy), alkylthio, cyano, halo, amino, and nitro. Such aryl radicals may be linked to the remaining portion of the molecule through any position on the ring or substituents that results in a stable compound having the desired activity. Examples of specific aryl groups are phenyl, methylphenyl, dimethylphenyl, aminophenyl, nitrophenyl, hydroxyphenyl, pyrrolyl, thiazolyl, oxazolyl, pyridyl, pyrimidinyl and the like.

The term “arylalkyl” means an alkyl radical substituted with one or more aryl substituents. The number of carbon atoms specified for arylalkyl radicals refers to the alkyl portion of the segment. Examples of specific arylalkyl segments include benzyl, methylbenzyl, dimethylbenzyl, aminobenzyl, nitrobenzyl, hydroxybenzyl, and the like.

The term “carbocyclyl” means a segment comprising one or more rings, each ring typically having 3-12, 3-8 or 3-6 ring members, which may be independently saturated, unsaturated, or aromatic and which contain only carbon ring members. “Carbocycl” includes moieties that are unsubstituted or substituted with one or more substituents, including (but not limited to) alkyl (preferably, lower alkyl), hydroxy, alkoxy (such as, lower alkoxy), alkylthio, cyano, halo, amino, and nitro. Suitable carbocycles for use in the compounds of this invention include (without limitation) phenyl, benzyl, indanyl, indenyl, naphthyl, tetralyl, decalyl, cyclopentyl, cyclopentenyl, cyclohexyl, cyclohexenyl and cycloheptyl. Specific carbocycles for this use include (without limitation) cycloalkyl, cycloalkenyl and mono- or bicyclic carbocyclic aromatic rings or ring systems containing from three to ten carbon atoms.

The term “contiguous linear connectivity” means connected together so as to form an uninterrupted linear array or series of atoms. For example, a linker of the compounds described herein having a specified number of atoms in contiguous linear connectivity has at least that number of atoms connected together so as to form an uninterrupted chain, but may also include additional atoms that are not so connected (for example, branches or atoms contained within a ring system).

The term “cycloalkyl” means a mono- or polycyclic alkyl radical.

“DNA polymerase III” (or “Pol III”) has the same meaning known and understood in the art and refers, therefore, to the DNA polymerase III that is involved in the replication of the bacterial chromosome and, therefore, is critical to normal growth of the bacterial cell. There are two classes of DNA polymerase, i.e., DNA polymerase IIIC (“pol IIIC” or “DNA pol IIIC”) and DNA polymerase IIIE (“pol IIIE” or “DNA pol IIIE”). Typical Gram-negative bacteria only possess species of the pol IIIE class of DNA polymerase III, whereas Gram-positive bacteria and mycoplasma bacteria possess DNA polymerase III species of both pol IIIE and pol IIIC classes. The pyrazole carboxylic acid hydrazide compounds described herein are capable of inhibiting one or more enzyme species of the pol IIIC and/or pol IIIE classes of DNA polymerase III.

“Gram-positive” and “Gram-negative” with respect to eubacteria have the meanings commonly understood to those familiar in the art for distinguishing bacteria based on the result of staining bacterial cells by the classic Gram stain. Gram-positive bacteria retain crystal violet dye of the classic Gram stain and appear blue under the microscope, whereas Gram-negative lose the crystal violet dye and will take up a red counterstain dye (e.g., safranin, acid fuchsin). It is well known in the art that the difference between the positive and negative results of a Gram stain is based on the difference in cell walls between Gram-positive and Gram-negative bacteria.

“Mycoplasma” and “mycoplasmata” have the microbiological meanings familiar to those skilled in art and refer to bacteria traditionally assigned to the genera Mycoplasma and Ureaplasma. Mycoplasma bacteria are technically Gram-negative as, unlike eubacteria, mycoplasma bacteria lack a cell wall and, thus, will not stain positive in the Gram stain. Owing to this distinct cellular morphology, mycoplasma bacteria are not considered representative of the typical Gram-negative eubacteria and, therefore, are referred herein separately with respect to the description of the compositions and methods of the invention.

“Halo” means a halogen radical, i.e., fluoro, chloro, bromo, or iodo.

“Heterocyclyl” means a heterocyclic radical containing one or more rings which may be saturated, unsaturated, or aromatic wherein at least one ring of the radical optionally contains one or more heteroatoms selected from nitrogen (N), oxygen (O), and sulfur (S) in one or more rings. Suitable heterocyclyl moieties for use in the compounds of this invention include radicals of (without limitation) furan, dioxolane, thiophene, pyrrole, pyrazole, triazole, imidazole, pyrrolidine, pyran, pyridine, pyrimidine, morpholine, piperidine, piperazine, oxazole, isoxazole, oxazoline, oxazolidine, oxathiazole, thiazole, isothiazole, thiadiazole, tetrazole, benzofuran, indole, isoindole, quinazoline, quinoline, isoquinoline, purine, pyrrolopyrimidine, pyrazolopyrimidine, pteridine, ketal. In addition, heterocyclyl radicals may contain one or more substituents (i.e., a ring substituent, such as a halogen atom, an alkyl radical, or aryl radical) attached to a ring member atom of the heterocyclyl radical. All stable isomers of heterocyclyl groups are contemplated in this definition.

“Individual” means an animal, including a mammal, such as a human. An individual that has, is suspected of having, or is at risk for a bacterial infection, may also be referred to as a “patient”.

“Linker” means a diradical having from 1-6 atoms in contiguous linear connectivity (i.e., as defined above and excluding atoms present in any side chains and branches), that covalently connects the phenyl portion of a compound of this invention to the pyrazole portion of the compound, as illustrated in formulas 1a and 1b. The atoms of the linker in contiguous linear connectivity may be connected by saturated or unsaturated covalent bonds. Linkers include, but are not limited to, alkylidene, alkenylidene, alkynylidene and cycloalkylidene (such as lower alkylidene, cycloalkylidene, alkylycloalkylidene and alkyl-substituted alkylidene) linkers wherein one or more (for example, between 1 and 4; such as 1 or 2) carbon atoms may be optionally replaced with O (oxygen), S (sulfur), or N (nitrogen) and wherein two or more (for example, 2-4 (such as 2 or 3)) adjacent atoms may be optionally linked together to form a carbocyclic or heterocyclic moiety within the linker (which may be monocyclic, polycyclic and/or fused, and which may be saturated, unsaturated, or aromatic). Examples of specific linkers useful in the compounds of the invention include (without limitation) diradicals of alkyl, alkenyl, alynyl, alkoxy, alkoxyalkyl, alkylaminoalkyl, cycloalkyl, alkylcycloalkyl, and alkyl-substituted alkylcycloalkyl (wherein one or more carbon atoms in any of these linkers may be optionally replaced with O, S, or N).

“Lower” means the group to which it is applied preferably has 1-6, and more preferably 1-4, carbon atoms, except in the case of rings (such as cycloalkyl), in which case “lower” signifies 3-6 ring member atoms. Unless otherwise noted to the contrary, all substituents herein to which the term “lower” is applicable, shall be preferred as such.

“Protecting group” means a chemical group that is known in the art to protect an otherwise reactive segment against undesirable reaction during one or more particular synthetic procedures and that is selectively removable under a given set of reaction conditions. Protecting groups may be suitable for use, for example, where a compound of the invention contains a free amino or carboxylic acid functionality. Suitable protecting groups for such use are well known to those of ordinary skill in the art and include, without limitation, trimethylsilyl, dimethylhexylsilyl, t-butyldimethylsilyl, t-butyldiphenylsilyl, trityl, alkyl groups, acyl groups (such as acetyl and propionyl), methanesulfonyl, and p-toluenesulfonyl. Protecting groups that are especially useful for protecting amide functionalities include (without limitation): aralkoxymethyl (for example, benzyloxymethyl and substituted benzyloxymethyl); alkoxymethyl (for example, methoxymethyl and trimethylsilylethoxymethyl); trialkyl/arylsilyl (for example, trimethylsilyl, t-butyldimethylsily, t-butyldiphenylsilyl); tri alkyl/arylsilyloxymethyl (for example, t-butyldimethylsilyloxymethyl, t-butyldiphenylsilyloxymethyl); 4-alkoxyphenyl (for example, 4-methoxyphenyl); 2,4-di(alkoxy)phenyl (for example, 2,4-dimethoxyphenyl); 4-alkoxybenzyl (for example, 4-methoxybenzyl); 2,4-di(alkoxy)benzyl (for example, 2,4-di(methoxy)benzyl); alk-1-enyl (for example, allyl, but-1-enyl and substituted vinyl for example, 2-phenylvinyl); allyloxycarbonyl; and lower alkoxycarbonyl and benzyloxycarbonyl. Examples of suitable protecting groups for carboxyl groups are the residue of an ester-forming aliphatic or araliphatic alcohol or of an ester-forming silanol (the alcohol or silanol preferably containing from 1-20 and, more preferably, from 1-10 carbon atoms). Protecting groups that are especially useful for protecting amino functionalities include, without limitation: acyl groups, including acetyl, trifluoroacetyl, benzoyl; and acyloxy groups, including t-butyloxycarbonyl, benzyloxycarbonyl, fluoroethenylmethoxycarbonyl, and the like. Protecting groups may be removed by standard methods after the contemplated reaction has been completed. For a more complete description of protecting groups and their use, see, T. W. Greene and P. G. M. Wuts, Protective Groups in Organic Synthesis. 2nd ed. (John Wiley & Sons, New York, 1991).

Pyrazole carboxylic acid hydrazide compounds may be shown to possess one or more properties that make these compounds useful in the invention. Such properties include the ability to inhibit the enzymatic activity of one or more species of DNA polymerase III and the ability to inhibit growth of one or more species of Gram-positive bacteria, Gram-negative bacteria, and/or mycoplasma bacteria. Methods of the invention for inhibiting the growth of bacteria involve providing a pyrazole carboxylic acid hydrazide compound described herein in an amount sufficient to be effective for inhibition. By “inhibiting” or “inhibition” of bacteria growth is meant reducing the cellular growth (population) by at least 80%. In certain embodiments, bacterial growth may be inhibited by 90%, 95%, or 99% or more. The degree of inhibition can be ascertained by an in vitro growth assay, for example, by standard liquid culture techniques or plating on solid media supplemented with a compound described herein. Compounds showing inhibition of colony formation at suitable MICs (minimal inhibitory concentrations), for example, less than 100 μg/ml, are typically considered as candidates for further examination as possible therapeutic agents useful to treat an individual that has a bacterial infection or is at risk of or is otherwise susceptible to a bacterial infection. In the context of inhibiting bacterial growth, by an “effective amount” of a compound is meant an amount which, when administered in vivo or in vitro, will achieve the above-stated levels of inhibition. In the context of “inhibiting” (or “inhibition of”) the activity of a DNA polymerase III enzyme, methods of the invention for inhibiting the activity of a DNA polymerase III enzyme involve contacting or incubating a DNA polymerase III (i.e., a pol IIIC or pol IIIE enzyme) in an assay for DNA polymerase III activity with a compound described herein such that the polymerase activity of the DNA polymerase III is diminished relative to the DNA polymerase III activity observed in absence of the compound.

A method for treating an individual with a bacterial infection involves or comprises administering to the individual a therapeutically effective amount of a pyrazole carboxylic acid hydrazide compound described herein. By “therapeutically effective amount” is meant an amount which, when administered to the individual in need, will alleviate one, some, or all of the symptoms of a bacterial infection. In the context of prophylaxis (i.e., preventative therapy or treatment), a “therapeutically effective amount” is an amount which, when administered to an individual, will prevent, inhibit, or reduce the likelihood of a bacterial infection from occurring in the individual. A particularly preferred “therapeutically effective amount” of a compound described herein eliminates completely (in a therapeutic treatment) or prevents completely (in a prophylactic treatment) bacterial infection in an individual. A therapeutically effective amount administered to an individual to treat a bacterial infection in that individual may be the same or different from a therapeutically effective amount administered prophylactically to the individual to prevent a bacterial infection.

By “an individual at risk or susceptible to a bacterial infection” is meant an individual (for example, a human or other mammal) that is at risk of contracting a bacterial infection. Examples of individuals at risk (i.e., susceptible to a bacterial infection) include, but are not limited to, those that have sustained wounds; those that have recently undergone a surgical procedure; those that are immunocompromised from disease (for example, individuals with acquired immunodeficiency syndrome (AIDS)); those that are immunocompromised from a medical treatment (for example, certain drugs, chemotherapies, radiation treatments); those that must work with such bacteria (as in clinical or research laboratories); and those (healthcare, public health, or related professions) that work in an environment containing bacterially contaminated materials or infected individuals. Individuals that may be “at risk of” or “susceptible to” a bacterial infection may also be readily assessed and identified using various clinical, diagnostic, environmental, and/or epidemiological procedures and devices available in the art.

A pyrazole carboxylic acid hydrazide compound described herein includes the corresponding “pharmaceutically acceptable salts of the compound”. By the term “pharmaceutically acceptable salts of the compound” as understood and used herein, is meant those salts of any pyrazole carboxylic acid hydrazide compound useful in the invention derived from an inorganic or organic acid or base recognized in the art that is compatible for pharmaceutical compositions. For convenience, the terms “pharmaceutical” and “pharmaceutically acceptable” also are understood to encompass compounds acceptable for the practice of veterinary medicine as well. Examples of suitable acids may include, without limitation, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids such as oxalic, while not themselves pharmaceutically acceptable, may be useful as intermediates in obtaining a compound useful in the invention and its pharmaceutically acceptable acid addition salts. Salts derived from appropriate bases may include, without limitation, alkali metal (for example, sodium, potassium), alkaline earth metal (for example, magnesium), ammonium, and NR¹¹₄⁺ (where R¹¹ is lower alkyl) salts, including choline, and the like. Reference, hereinafter, to a compound useful in the invention (or an equivalent term) is understood to include any and all corresponding pharmaceutically acceptable salts thereof.

By “substituted” is meant that one or more hydrogen atoms of a compound or portion of a compound are replaced by substituents typical for that type of compound or portion of compound, including, but not limited to, C₁₋₆ alkyl, C₃₋₆ cycloalkyl, acyl, hydroxyl, oxo, C₁₋₆ alkoxyl, amino, carboxyl, halo, cyano, azido, nitro, C₆₋₁₂ aryl, C₆₋₁₂ arylalkyl, C₆₋₁₂ heteroaryl, (CO)—C₁₋₆ alkyl, (CO)—C₆₋₁₂ aryl, (SO₂)—C₁₋₆ alkyl, (SO₃)—C₁₋₆ alkyl, (SO₂)—C₆₋₁₂ aryl, (SO₃)—C₆₋₁₂ aryl, (SO₂)—C₆₋₁₂ heteroaryl, and (SO₃)—C₆₋₁₂ heteroaryl. The substituents can in turn be substituted with functional groups, including, but not limited to, halo, trifluoromethyl, hydroxyl, and carboxyl.

The compounds useful in this invention may contain additional functional groups that may increase the water solubility of the compounds thereby facilitating bioavailability, absorption, and distribution of the compounds in humans and other animals, without interfering with the property to inhibit undesired growth of one or more strains or species of bacteria. Alternatively, the compounds themselves may be relatively water-soluble or may form salts that are relatively water-soluble.

The low toxicity of the compounds described herein to mammals and other animals endows these compounds with the type of characteristic(s) particularly desired for use as therapeutically effective antibacterial compounds. Furthermore, compounds described herein target one or more essential enzymes in bacterial DNA replication that have not been a target for any previously marketed or widely used antibiotic; development of drug resistance will thus be minimized. The compounds may thus be used to circumvent natural and acquired resistance of pathogenic bacteria to conventional antimicrobials.

Unless otherwise specified, the terms defined above shall have the meanings ascribed above even when such terms are used as a part (for example, as a prefix or a suffix) of a different term (for example, the definition of “alkyl” given above shall apply to the alkyl portion of an “alkylamino” group). Furthermore, unless otherwise defined, all technical and scientific terms used herein that are not defined have the same meaning as commonly understood by one of ordinary skill in the art to which this invention belongs. Specific examples of chemical groups falling within the general categories described above are provided for illustration or convenience and are not meant to be viewed as exhaustive or limiting to the scope of the invention in any way. Any radical defined above as being optionally substituted may be linked directly or indirectly through any of its substituents. Combinations and choices of substituents shall be selected so as to produce stable chemical compounds having the desired antibacterial activity and which are available by conventional synthetic techniques. For any given substituent, stated preferences or selections will apply even if that substituent is used in a different combination of variables. In all cases, functional oxygen, nitrogen, sulfur, or other chemically active segments may be protected as necessary or desired using conventional protecting groups. In some cases, a compound may exist in tautomeric forms, for example, as imine-enamine forms, as when R⁴ is hydrogen (H) in formulas 1a and 1b (see, below). For compounds useful in the invention having one or more chiral centers, such compounds may be stereochemically pure, for example, individual enantiomers or diastereomers, or may be present as a mixture of stereoisomers, such as a racemic or other ratio mixture of individual enantiomers or diastereomers. Such choices will be made on a case-by-case basis, taking into account the observed activity of the mixture and of individual stereoisomers.

Although methods and materials similar or equivalent to those described herein can be used in the practice or testing of the present invention, suitable methods and materials are described below. In case of conflict, the present specification, including definitions, will control. In addition, the materials, methods, and examples set forth herein are illustrative only and not intended to be limiting in any way.

Compounds Useful in the Invention

Representative species of the pyrazole carboxylic acid hydrazide compounds described herein were initially identified by screening a library of synthetic organic molecules for the ability to inhibit the activity of Gram-positive DNA pol IIIC and IIIE enzymes. Additional examples of these compounds were subsequently purchased from commercial vendors and examined for the ability to inhibit activity of one or more species of pol IIIC and/or pol IIIE enzymes and to inhibit growth of various bacterial strains and species.

The invention features methods and compositions comprising pyrazole carboxylic acid hydrazide compounds of formulas 1a and 1b shown below: 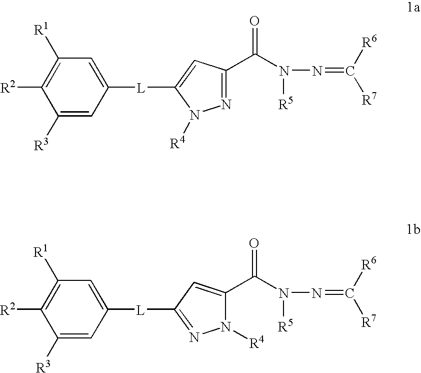
wherein R¹, R², and R³ are each, independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;

R⁴ and R⁵ are, independently selected from the group consisting of H, substituted or unsubstituted alkyl, substituted or unsubstituted alkenyl, substituted or unsubstituted alkynyl, and substituted or unsubstituted cycloalkyl wherein it is understood that, when R⁴ is H, two tautomeric forms depicted by structures 1a and 1b may exist;

R⁶ and R⁷ are, independently, H, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted alkylaryl, substituted or unsubstituted heteroaryl, or R⁶ and R⁷ are atoms that form part of a aromatic or non-aromatic, heterocylic or carbocyclic ring or ring system, comprising either a monocyclic ring or a fused ring system, having ring atoms selected from the group consisting of substituted or unsubstituted carbon, nitrogen or sulfur; and

L may be absent or selected from the group consisting of linkers having 1-6 atoms in contiguous linear connectivity;

or an enantiomeric or diastereomeric form or a pharmaceutically acceptable salt thereof.

In one embodiment, with respect to formulas 1 and 2, above, R¹, R², and R³ are each, independently selected from the group consisting of H, substituted or unsubstituted C₁₋₆ alkyl, substituted or unsubstituted C₂₋₆ alkenyl, substituted or unsubstituted C₂₋₆ alkynyl, halo, amino, substituted or unsubstituted acyl, substituted or unsubstituted aryl, substituted or unsubstituted C₁₋₆ alkylamino, substituted or unsubstituted C₁₋₆ alkylaminoalkyl, substituted or unsubstituted C₁₋₆ alkoxy, substituted or unsubstituted C₁₋₆ alkoxyalkyl, substituted or unsubstituted C₁₋₆ alkylthio, substituted or unsubstituted C₁₋₆ alkylthioalkyl, nitro, hydroxyl, cyano, substituted or unsubstituted C₃₋₁₂ carbocyclyl, substituted or unsubstituted C₃₋₁₂ carbocyclylalkyl, substituted or unsubstituted C₃₋₁₂ carbocyclyloxy, substituted or unsubstituted C₃₋₁₂ carbocyclyloxyalkyl, substituted or unsubstituted C₃₋₁₂ carbocyclylamino, substituted or unsubstituted C₃₋₁₂ carbocyclylaminoalkyl, substituted or unsubstituted 3-12 membered heterocyclyl, substituted or unsubstituted 3-12 membered heterocyclyl-C₁-C₆ alkyl, substituted or unsubstituted 3-12 membered heterocyclyloxy, substituted or unsubstituted 3-12 membered heterocyclyloxy-C₁-C₆-alkyl, substituted or unsubstituted 3-12 membered heterocyclylamino, and substituted or unsubstituted 3-12 membered heterocyclylamino-C₁-C₆-alkyl;

R⁴ and R⁵ are, independently selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl wherein it is understood that, when R⁴ is H, two tautomeric forms depicted by structures 1a and 1b may exist;

R⁶ and R⁷ are, independently, H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, substituted or unsubstituted C₄₋₁₂ heteroaryl, or R⁶ and R⁷ are atoms that form part of a aromatic or non-aromatic, heterocylic or carbocyclic ring or ring system, comprising either a 3-6 membered monocyclic ring or a 6-12 membered fused ring system, having ring atoms selected from the group consisting of substituted or unsubstituted carbon, nitrogen or sulfur; and

L may be absent or selected from the group consisting of linkers having 1-6 atoms in contiguous linear connectivity;

or an enantiomeric or diastereomeric form or a pharmaceutically acceptable salt thereof.

In another embodiment with respect to formulas 1 and 2, above, R¹, R², and R³ are each, independently selected from the group consisting of H, substituted or unsubstituted C₁₋₆ alkyl, substituted or unsubstituted C₂₋₆ alkenyl, substituted or unsubstituted C₂₋₆ alkynyl, halo, amino, substituted or unsubstituted acyl, substituted or unsubstituted aryl, substituted or unsubstituted C₁₋₆ alkylamino, substituted or unsubstituted C₁₋₆ alkylaminoalkyl, substituted or unsubstituted C₁₋₆ alkoxy, substituted or unsubstituted C₁₋₆ alkoxyalkyl, substituted or unsubstituted C₁₋₆ alkylthio, substituted or unsubstituted C₁₋₆ alkylthioalkyl, nitro, hydroxyl, cyano, substituted or unsubstituted C₃₋₁₂ carbocyclyl, substituted or unsubstituted C₃₋₁₂ carbocyclylalkyl, substituted or unsubstituted C₃₋₁₂ carbocyclyloxy, substituted or unsubstituted C₃₋₁₂ carbocyclyloxyalkyl, substituted or unsubstituted C₃₋₁₂ carbocyclylamino, substituted or unsubstituted C₃₋₁₂ carbocyclylaminoalkyl, substituted or unsubstituted 3-12 membered heterocyclyl, substituted or unsubstituted 3-12 membered heterocyclyl-C₁-C₆ alkyl, substituted or unsubstituted 3-12 membered heterocyclyloxy, substituted or unsubstituted 3-12 membered heterocyclyloxy-C₁-C₆-alkyl, substituted or unsubstituted 3-12 membered heterocyclylamino, and substituted or unsubstituted 3-12 membered heterocyclylamino-C₁-C₆-alkyl;

R⁴ and R⁵ are, independently selected from the group consisting of H, substituted or unsubstituted C₁₋₆ alkyl and substituted or unsubstituted C₃₋₈ cycloalkyl wherein it is understood that, when R⁴ is H, two tautomeric forms depicted by formulas 1a and 1b may exist;

R⁶ and R⁷ are, independently, H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, substituted or unsubstituted C₄₋₁₂ heteroaryl, or R⁶ and R⁷ are atoms that form part of a ring system having the structure: 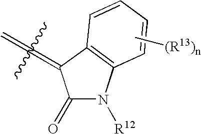
wherein R¹² is selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl,

each R¹³ is selected from the group consisting of substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl,

n is an integer selected from 0, 1, 2, 3, and 4; and

L may be absent or selected from the group consisting of linkers having 1-3 atoms in contiguous linear connectivity;

or an enantiomeric or diastereomeric form or a pharmaceutically acceptable salt thereof.

Further embodiments of this invention include those wherein R¹, R², and R³ are each independently selected from the group consisting of H, substituted or unsubstituted C₁₋₆ alkyl, substituted or unsubstituted C₂₋₆ alkenyl, substituted or unsubstituted C₂₋₆ alkynyl, halo, amino, substituted or unsubstituted acyl, substituted or unsubstituted C₁₋₆ alkoxy, substituted or unsubstituted C₁₋₆ alkoxyalkyl, nitro, hydroxyl, cyano, substituted or unsubstituted C₃₋₈ carbocyclyl, substituted or unsubstituted C₃₋₈ carbocyclylalkyl, substituted or unsubstituted C₃₋₈ carbocyclyloxy, substituted or unsubstituted C₃₋₈ carbocyclyloxyalkyl, substituted or unsubstituted 3-8 membered heterocyclyl, substituted or unsubstituted 3-8 membered heterocyclyl-C₁-C₆ alkyl, substituted or unsubstituted 3-8 membered heterocyclyloxy, substituted or unsubstituted 3-8 membered heterocyclyloxy-C₁-C₆-alkyl, substituted or unsubstituted 3-12 membered heterocyclylamino, and substituted or unsubstituted 3-12 membered heterocyclylamino-C₁-C₆-alkyl,

R⁴, R⁵, and R⁶ are H, and R⁷ is phenyl optionally substituted by one or more C₁₋₆ alkyl, C₁₋₆ alkoxy, halo, amino, hydroxy, halo or R⁶ and R⁷ are atoms that form part of a ring system having the structure: 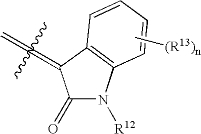
wherein R¹² is selected from the group consisting of H, substituted or unsubstituted C₁₋₆ alkyl and substituted or unsubstituted C₃₋₈ cycloalkyl, and

each R¹³ is selected from the group consisting of substituted or unsubstituted C₁₋₆ alkyl, substituted or unsubstituted C₂₋₆ alkenyl, substituted or unsubstituted C₂₋₆ alkynyl, halo, amino, substituted or unsubstituted C₁₋₆ acyl, substituted or unsubstituted aryl, substituted or unsubstituted C₁₋₆ alkoxy, substituted or unsubstituted C₁₋₆ alkoxyalkyl, nitro, hydroxyl, and cyano,

n is an integer selected from 0, 1, and 2; and

L is absent; or

an enantiomeric or diastereomeric form or a pharmaceutically acceptable salt thereof.

Other particular embodiments of this invention include compounds of formula 2: 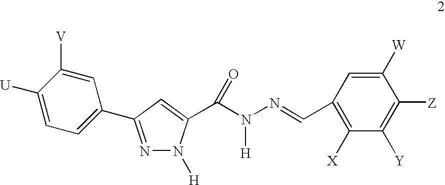
wherein U, V, W, X, Y, and Z may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl; or an enantiomeric or diastereomeric form or a pharmaceutically acceptable salt thereof. Another embodiment of this invention includes the compounds of formula 2, wherein U, V, W, X, Y and Z are independently selected from the group consisting of H, C₁₋₄ alkyl, C₃₋₆ cycloalkyl, C₁₋₄ alkoxy, halo, hydroxyl, and amino; or an enantiomeric or diastereomeric form or a pharmaceutically acceptable salt thereof.

A further embodiment of this invention includes compounds of formula 3: 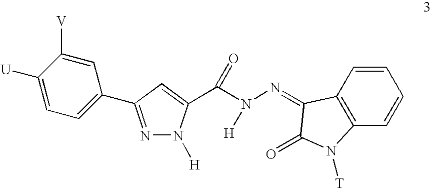
wherein, U and V may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl; and

T is selected from the group consisting of H, substituted or unsubstituted alkyl, substituted or unsubstituted alkenyl, substituted or unsubstituted alkynyl, and substituted or unsubstituted cycloalkyl; or

an enantiomeric or diastereomeric form or a pharmaceutically acceptable salt thereof.

A further embodiment of this invention includes compounds of formula 3, wherein U and V are independently selected from the group consisting of H, C₁₋₄ alkyl, C₁₋₄ alkoxy, and aryl, and T is selected from the group consisting of H and C₁₋₄ alkyl; or an enantiomeric or diastereomeric form or a pharmaceutically acceptable salt thereof.

Additional embodiments of this invention may be readily made by combining portions of any of the above embodiments with portions of one or more other embodiments. For example, with respect to pyrazole carboxylic acid hydrazide compounds used in methods and compositions of the invention, it is clear that formulas 1A and 1B not only permit each R group to be independently selected from a particular group of preferred individual substituents or ligands, but also that various combinations of such independent R groups may be used to make a variety of pyrazole carboxylic acid hydrazides encompassed by the invention. In addition, other embodiments are described herein that will be evident from the further description, examples, and claims below.

As explained in more detail below, the properties of the pyrazole carboxylic acid hydrazide compounds described herein permit the use of these compounds in a variety of methods and compositions. For example, a pyrazole carboxylic acid hydrazide compound described herein may be used to treat an individual (for example, a human or other animal) having or at risk of having a bacterial infection comprising administering to the individual a therapeutically effective amount of the compound. Pyrazole carboxylic acid hydrazide compounds described herein may also be used in biochemical assays or studies where there is a need for a specific inhibitor of one or more species of DNA polymerase III (for example, particular species of pol IIIC and/or pol IIIE). Yet other uses of the compounds described herein include the use as inhibitors of bacterial growth not only in vivo, but also in vitro, as in preventing bacterial growth in various cell and tissue culture media, compositions, and solutions used in research and manufacturing. Furthermore, as with many antibiotics, the antibacterial activity of compounds described herein permits their use under appropriate circumstances to alter the endogenous microflora of an individual in order to promote better health or growth of the individual, even in the absence of clinical symptoms of infection by pathogenic bacteria.

The compounds described herein also may have special advantages in the treatment of microorganisms that have or may become resistant to currently used antibiotics. For example, these compounds may inhibit one or more DNA polymerase III enzymes from strains or species of a variety of Gram-negative, Gram-positive, or mycoplasma bacteria. Inhibition of DNA polymerase III, a family of polymerase enzymes responsible for replication of the genomes of most if not all bacteria, causes inhibition of bacterial growth and may also cause bacterial cell death. With respect to Gram-positive bacteria, particularly preferred is the use of compositions and methods of the invention to inhibit or prevent growth of one or more strains or species of such clinically relevant Gram-positive bacteria as those selected from the group consisting of Streptococcus, Enterococcus, Staphylococcus, Bacillus, Clostridium, Listeria, and combinations thereof. With respect to Gram-negative bacteria, particularly preferred is the use of compositions and methods described herein to inhibit or prevent growth of one or more strains or species of such clinically relevant Gram-negative bacteria as those selected from the group consisting of Escherichia, Salmonella, Pseudomonas, Helicobacter, Legionella, Shigella, Yersinia, Neisseria, and combinations thereof.

Other uses of the compounds and compositions according to this invention will be apparent to those of ordinary skill in the art and are expressly included as a part of this invention.

Mechanism of Action

Genome sequence analysis has indicated that organisms such as the mycoplasma bacteria (mycoplasmata) and Gram-positive eubacteria of the so-called low G:C class, i.e., those with genomes containing a proportion of guanine+cytosine (G+C) of less than 0.5, contain two types or classes of DNA polymerase III (pol III): pol IIIC, encoded by a polC gene, and pol IIIE, encoded by one or more dnaE genes, (see, Wright and Brown, Current Opinion in Anti-Infective Investigational Drugs, 1: 45-48 (1999); Braithewaite and Ito, Nucl. Acids Res., 21: 787-802 (1993)). Typical Gram-negative bacteria only possess a pol IIIE class of DNA polymerase III for replicative chromosomal DNA synthesis. The compounds described herein specifically inhibit the activity of one or more species of pol IIIC and/or pol IIIE enzymes.

Without wishing to be bound by any particular theory or mechanism, the compounds described herein appear to mimic or otherwise compete with one or more deoxyribonucleoside-5-triphosphates (dNTPs), thereby physically inhibiting one or more species of DNA polymerase III enzymes as noted above. Unexpectedly, enzyme kinetic analyses indicate that these compounds appear to act as uncompetitive inhibitors (see, Example 3, below). Furthermore, the ability of the pyrazole carboxylic acid hydrazide compounds described herein to inhibit the activity of a DNA polymerase III is particularly surprising as none of these compounds possess any of the heterocyclic nitrogenous bases (i.e., guanine, cytosine, adenine, thymine, uracil) that are normally used in the synthesis or replication of DNA or RNA and that, if present, may have suggested a more classic mechanism of polymerase inhibition.

Whatever the exact mechanism for action, a particular pyrazole carboxylic acid hydrazide compound described herein may have a relatively broad- or narrow-spectrum activity, i.e., with respect to inhibiting growth of one or more bacterial species or strains, based at least in part on the ability to inhibit one or more species of bacterial DNA polymerase III. Other factors that may affect the spectrum of activity of a compound to inhibit the growth of one or more bacterial species or strains include, but are not limited to, the ability of the compound to pass through the bacterial cell membrane or wall, the ability to resist inactivation within a bacterial cell and/or bacterial periplasmic space (as in Gram-negative bacteria), the relative solubility of the compound in aqueous solutions or body fluids, and the in vivo half-life of the compound when administered to an individual by a particular route.

Synthesis and Characterization of Antibacterial Compounds Useful in the Invention

The compounds useful in the compositions and methods of this invention are pyrazole carboxylic acid hydrazides and structurally related compounds as described in formulas 1a and 1b (see, above). Some of these compounds may be obtained from a commercial source, however, the compounds may also be produced using standard synthetic procedures known to those skilled in organic chemistry synthesis and/or following the synthetic schemes below.

Compounds described herein include without limitation 5-phenylpyrazole-3-carboxylic acid benzalhydrazides, where each of the phenyl rings may be optionally and independently substituted with one or more (for example, 2-4 or 2-3) phenyl substituents such as those included in the definitions of R¹ in formulas 1a and 1b. Particularly useful are compounds of formulas 1a and 1b, having the particular structure shown in formula 2 and the substituents listed in Table 1, below:

TABLE 1

| | |
|---|---|
| | 2 |
| 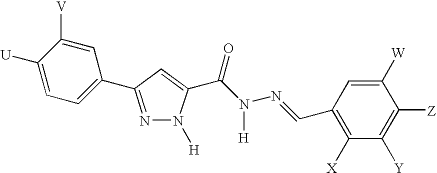 | |

| Compound | | | | | | |
|---|---|---|---|---|---|---|
| Number | U | V | W | X | Y | Z |
| 1 | H | H | OCH₃ | H | OCH₃ | OCH₃ |
| 2 | H | OCH₃ | H | H | OCH₃ | OCH₃ |
| 3 | H | OCH₃ | H | H | OCH₂CH₃ | OH |
| 4 | H | H | H | OH | H | OH |
| 5 | H | H | H | H | OCH₃ | OH |
| 6 | H | H | Br | OH | H | H |
| 7 | H | OCH₂CH₃ | H | OH | OCH₃ | H |
| 8 | CH₃ | H | H | OH | OCH₃ | H |
| 9 | H | H | H | OH | H | H |
| 10 | H | OCH₃ | H | H | OH | H |
| 11 | H | H | H | H | H | CH₃ |
| 12 | H | H | C₄H₉ | H | C₄H₉ | OH |
| 13 | OCH₃ | H | H | H | H | H |
| 14 | H | OCH₃ | H | H | H | OH |
| 15 | Cl | H | H | H | OCH₃ | OCH₃ |
| 16 | OCH₃ | H | H | H | H | OH |
| 17 | OCH₃ | H | OCH₃ | H | OCH₃ | OCH₃ |
| 18 | OCH₃ | H | H | H | OCH₃ | OCH₃ |
| 19 | OC₂H₅ | H | H | H | OCH₃ | OH |

Other examples of pyrazole carboxylic acid hydrazides useful in this invention are represented by formula 3, shown below. Members of this class include the compounds having substituents listed in Table 2, below:

TABLE 2

| | |
|---|---|
| | 3 |
| 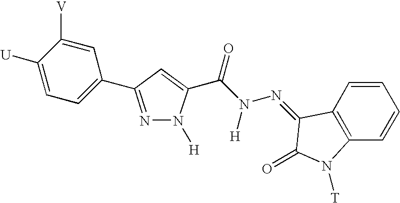 | |

| Compound Number | T | U | V |
|---|---|---|---|
| 20 | C₂H₅ | H | H |
| 21 | C₂H₅ | H | C₆H₅ |

Methods of Preparing Compounds Useful in the Invention
Step 1. Synthesis of 5-phenyl-3-aminopyrazoles.

There are several known synthetic routes to substituted 5-phenyl-3-aminopyrazoles (see, for example, Kost, N., Adv. Heterocycl Chem., Vol. 6 (Wiley, New York, 1967); Fusco, R. Pyrazoles, Pyrazolines, Indazolines and Condensed Rings, (Interscience, New York, 1967); Ege, G. and Franz, H., J. Heterocycl. Chem. 19: 1267-1273 (1982); Coan, B. and Becker, E. I. J. Amer. Chem. Soc. 76: 501 (1954); Fusco et al., Il Farmaco (Sci.), 10: 919-944 (1968); Gadekar et al., J. Med. Chem. 3: 616-618 (1968)). Scheme I illustrates two synthetic routes. 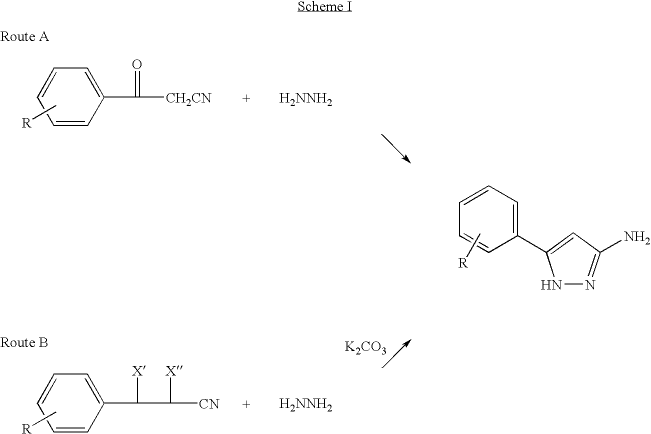
In Scheme I, above, R may be absent or represent up to 5 substituents, which may be the same or different, and X′ and X″ are each, independently, halo.

Route A uses β-ketonitriles while Route B uses 2,3-dihalo-3-phenylpropanenitriles, both routes taking advantage of commercially available intermediates.

Step 2. Synthesis of substituted 5-phenylpyrazole-3-carboxylic Acid Chlorides.

The carboxylic acid ester derivatives of pyrazole are synthesized from β-keto esters and α-halohydrazones (Scheme II, Route A). The esters are hydrolyzed to the acids, and the latter are converted to the acid chlorides by standard methods well known to those trained in the art of organic synthesis. Alternately (Scheme II, Route B), the target pyrazole carboxylic acids can be prepared by hydrolysis of the 3-cyanopyrazole derivative, which is prepared in turn from the 3-aminopyrazoles as described in Step 1. and Scheme I (above), by using standard methods well known to those trained in the art of organic synthesis. 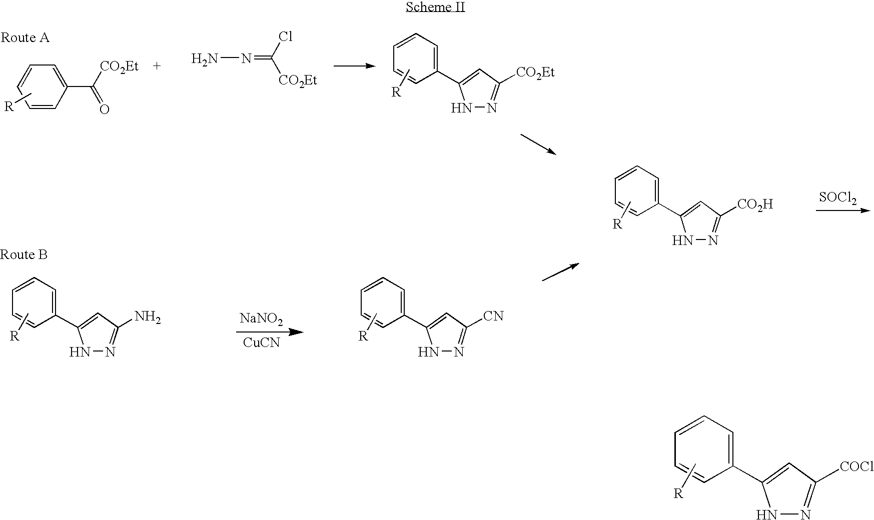
In Scheme II, above, R is (are) as described above.
Step 3. Synthesis of Substituted Hydrazones.

The synthesis of substituted hydrazones is accomplished by the reaction between hydrazine hydrate and substituted aldehydes or ketones. The chemistry is done under standard literature conditions. There are a large number of commercially available aldehydes and ketones, and the preparation of other non-commercially available ones may be undertaken using known synthetic methods.

Step 4. Synthesis of Pyrazole Carboxylic Acid Hydrazide Compounds Useful in the Invention.

a. Coupling of the Substituted 5-phenylpyrazole-3-carboxylic Acid Chlorides with Substituted Hydrazones.

The 5-phenylpyrazole-3-carboxlic acid chlorides (from Step 2., above) are treated with the substituted hydrazones (from Step 3., above) under standard reaction conditions (Scheme III). The general method (Scheme III, A.) is done with standard base catalysts. Preparation of benzalhydrazone (Scheme III, B.) and heterocyclylhydrazone (Scheme III, C.) compounds of the invention is also illustrated below. 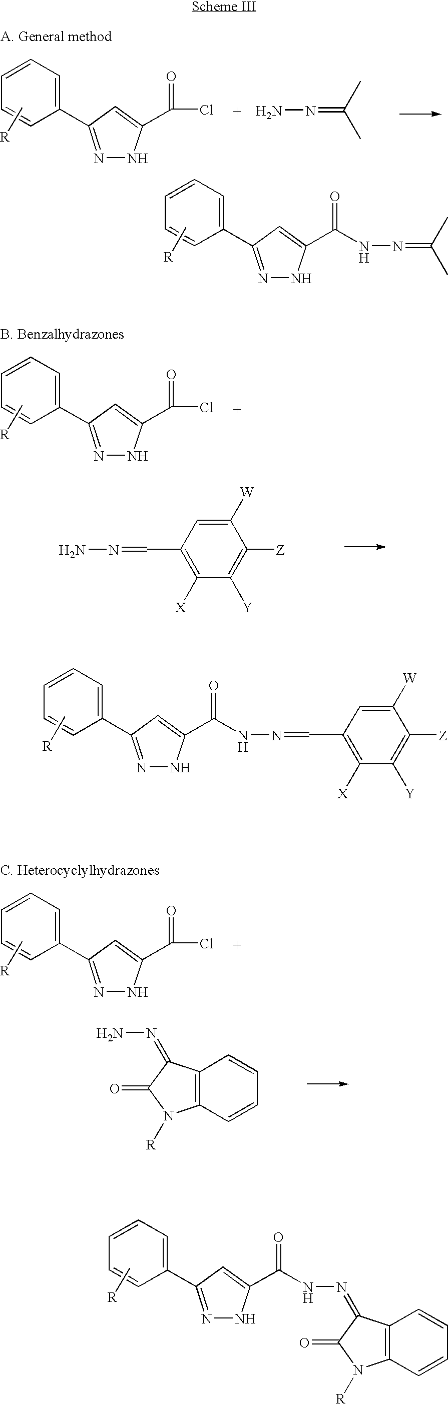
R is (are) as described above, and W, X, Y, and Z have the definitions set forth above.
b. Coupling of Substituted 5-phenylpyrazole-3-carboxylic Acid Hydrazides with Substituted Aldehydes and Ketones.

An alternate method involves conversion of the substituted 5-phenylpyrazole-3-carboxylic acid chlorides (from Step 2., above) to the corresponding substituted acid hydrazide, and condensation of the latter with substituted aldehydes and ketones (Scheme IV). 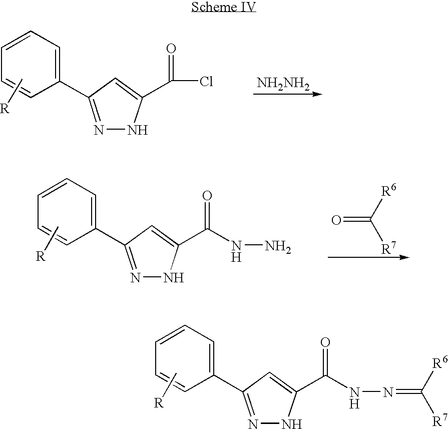
where R is (are) as described above.
Compound Efficacy in Inhibiting DNA Polymerase IIIC or IIIE Activity

The ability of a particular pyrazole carboxylic acid hydrazide compound to inhibit the activity of one or more species of a DNA pol IIIC or pol IIIE can be readily determined using known in vitro assays for DNA polymerase activity (see, for example, DNA polymerase assays described in Barnes and Brown, Nucl. Acids Res., 6: 1203-1219 (1979); Trantolo et al., J. Med. Chem., 29: 676-681 (1986); Mills et al., J. Bacteriol., 132: 641-649 (1977); and Low et al., J. Biol. Chem., 251: 1311-1325 (1976); all incorporated herein by reference). The species of pol IIIC or pol IIIE employed in a such an assay may be obtained from a commericial source, purified from a bacterial species of interest, and/or produced recombinantly using standard recombinant DNA technology. By including a test pyrazole carboxylic acid hydrazide compound in a side-by-side assay with a control polymerase reaction (i.e., no added test compound), the effect of the test compound on polymerase activity can be readily assessed. Test compounds with an observable or otherwise desired level of inhibition of the natural or recombinant bacterial DNA polymerase IIIC or IIIE are candidate therapeutics for further evaluation.

Detection of Bacterial Infection

A skilled healthcare professional may initially suspect that an individual suffers from (or is at risk of) a bacterial infection by physical examination and interviewing the individual for symptoms or possible exposure to such bacteria. Biological and biochemical evidence of infection by one or more bacterial strains or species may be obtained by analyzing a tissue or fluid sample from an individual using any of a variety standard clinical diagnostic methods, including but not limited to, plating bacteria from a sample on selective or diagnostic media known in the art, Gram staining bacteria present in a sample, and assaying a sample from an individual for bacteria using a commercially available diagnostic strip or device (for example, API diagnostic apparatuses, bioMerieux, Durham, N.C.). If time permits, bacteria isolated from a sample of an individual may be further tested for sensitivity to (i.e., growth inhibition) a spectrum of various known antibiotics or compounds described herein by incubating the bacteria in a series of liquid media or on plates of solid media that have been supplemented with a particular known antibiotic or compound described herein.

Compositions, Uses, Routes of Administration

Pyrazole carboxylic acid hydrazide compounds and compositions comprising these compounds as described herein have a variety of therapeutic (i.e., medical and veterinary therapy) and non-clinical uses based on the biochemical property to inhibit activity of DNA polymerase III. For example, methods and compositions (or “formulations”) provide new therapies to treat or prevent (prophylaxis) bacterial infections in an individual, including infections caused by bacterial strains and species that are resistant to previously known antibiotics. Prophylactic treatment using a compound described herein may be particularly appropriate for immunocompromised individuals or for individuals following surgery or dental procedures. Lists of relevant conditions for application of the methods and compositions of the invention is not intended to be limiting, and any appropriate infection responsive to a pyrazole carboxylic acid hydrazide compound may be treated using one or more methods or compositions described herein.

Therapeutic compositions and methods of the invention may be used to not only treat or prevent bacterial infections in humans, but other animals that have or at risk of a bacterial infection, including pets, livestock, research animals, undomesticated animals, and zoo animals, for example, pigs, cows, horses, goats, sheep, chickens, turkeys, rats, mice, rabbits, non-human primates, marine mammals, and fish. The therapeutic compositions and methods of the invention are useful in treating bacterial infections in an individual caused by a species or strain of Gram-positive bacteria, mycoplasma bacteria, and/or Gram-negative bacteria.

Compounds and compositions of the invention may also be used in non-therapeutic settings where prevention of bacterial contamination is required, such as to eliminate or prevent bacterial growth in various cell or tissue culture media and other solutions and compositions used in research and manufacturing.

Compounds and compositions of the invention may also be used in biochemical studies as specific inhibitors of one or more species of DNA polymerase III.

The pyrazole carboxylic acid hydrazide compounds described herein may be formulated for pharmaceutical, veterinary, and tissue or cell culture use, optionally together with an acceptable diluent, carrier, or excipient and/or in unit dosage form. In using the compounds described herein, conventional pharmaceutical, veterinary, or culture practice can be employed to provide suitable formulations or compositions, all of which are encompassed by the pharmaceutical compositions of this invention. In addition, compositions comprising an antibacterial pyrazole carboxylic acid hydrazide compound described herein may be prepared using procedures and ingredients as similarly employed in preparing pharmaceutical compositions comprising known antibiotics (such as erythromycin or other known antibiotic).

While it is possible that, for use in therapy, a compound described herein may be administered as the raw chemical compound (i.e., administration of a “composition” which consists of the compound alone), it is preferable to present the compound as an active ingredient in a pharmaceutical composition. The invention thus further provides a pharmaceutical composition comprising a compound described herein or a pharmaceutically acceptable salt thereof together with one or more pharmaceutically acceptable carriers and, optionally, other therapeutic or beneficial agents, such as, another antibiotic, antiviral compound, anti-cancer compound, vitamin, trace metal supplement, or ions to restore or maintain proper ionic balance in blood or other tissues. More particularly, examples of suitable therapeutic agents that may be used in combination with a compound described herein include, without limitation, penicillins and other beta lactamase inhibitors, carbapenems, cephalosporins, macrolides (including erythromycin and ketolides), sulfonamides, aminoglycosides, quinolones (such as fluoroquinolones), oxazolidinones, lipopeptides (such as daptomycin), tetracyclines, vancomycin, erythromycin, streptomycin, efflux pump inhibitors, lactoferrins, and cationic peptides. Such agents may be administered together with or separately from the compounds of this invention. In addition, certain individuals may suffer from or may be susceptible to simultaneous infections from bacteria and one or more viruses. Those individuals may benefit from simultaneous or separate co-administration of a compound or composition according to this invention and an anti-viral agent or medicament, for example, without limitation, an anti-influenza medication such as RELENZA® (zanamivir; GlaxoSmithKline, Research Triangle Park, North Carolina) and TAMIFLU® (oseltamivir phosphate; Roche Laboratories, Nutley, N.J.) or an anti-enteric virus drug such as pleconaril. Additional combination therapies may also include a compound of this invention and an anti-fungal agent, such as CANCIDAS® (caspofungin acetate; Merck, Whitehouse Station, N.J.), DIFLUCAN® (fluconazole; Pfizer, New York, N.Y.), and MYCOSTATIN® (nystatin; Bristol-Myers Squibb, New York, N.Y.). Clearly, the methods and compositions of the invention and various combination therapies described herein are merely exemplary and are not meant to limit possibilities for other combination treatments or co-administration regimens.

A pharmaceutically acceptable carrier(s) used in the pharmaceutical compositions of the invention must be “acceptable” in the sense of being compatible with (not destroying beneficial properties of) the other compounds, agents, and ingredients of the formulated pharmaceutical composition and not prohibitively deleterious to the individual to whom the pharmaceutical composition is administered. In liquid compositions, a pharmaceutically acceptable carrier may be a buffer solution, such as a phosphate buffered saline or another pharmaceutically acceptable, especially an isotonic, aqueous buffer. Pharmaceutically acceptable liquid compositions can, for example, be prepared by dissolving or dispersing a compound described herein and, optionally, one or more pharmaceutical adjuvants in an excipient, such as, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like.

The compositions of this invention may be administered to an individual parenterally or non-parenterally, including but not limited to, intravenously, subcutaneously, intramuscularly, intraorbitally, ophthalmically, intraventricularly, intracranially, intracapsularly, intraspinally, intracisternally, intraperitoneally, intranasally, by aerosol, by scarification, orally, buccally, rectally, vaginally, topically, and combinations thereof. The compositions of this invention may also be administered by the use of surgical implants that release a compound described herein, either as a bolus or slowly over a pre-selected period of time.

Without limitation, parenteral formulations may be, for example, in the form of liquid solutions or suspensions; for oral administration, formulations may be, for example, in the form of tablets, capsules, liquid solutions, and suspensions (wherein such solutions and suspensions may be particularly for formulations intended for pediatric use); and for intranasal administration, the formulations may be, for example, in the form of powders, nasal drops, or aerosols. Formulations for parenteral administration may contain as excipients sterile water or saline, polyalkylene glycols such as polyethylene glycol, oils of vegetable origin, hydrogenated naphthalenes, or biocompatible, biodegradable lactide polymers. Polyoxyethylene-polyoxypropylene copolymers can be used to control the release of the components of such compositions. Formulations for parenteral administration may also include glycocholate for buccal administration, methoxysalicylate for rectal administration, or citric acid for vaginal administration. Other potentially useful parenteral delivery systems for the compounds of the invention include ethylene-vinyl acetate copolymer particles, osmotic pumps, implantable infusion systems, and liposomes. Formulations for inhalation may contain lactose as an excipient, or may be aqueous solutions containing, for example, polyoxyethylene-9-lauryl ether, glycocholate and deoxycholate, or can be oily solutions for administration in the form of nasal drops, or can be gels to be applied intranasally. Other suitable formulations for parenteral, oral, or intranasal delivery of the compounds of this invention will be well known to those of ordinary skill in the art.

Methods well known in the art for preparing and formulating various pharmaceutical compositions may also be found in standard texts, for example, Remington's Pharmaceutical Sciences (Martin, E. W. (ed.) latest edition Mack Publishing Co., Easton, Pa.).

For topical administration, formulations and dosages may be similar to those used for other antibiotic drugs, for example, erythromycin. Topical administration of the pharmaceutical compositions of this invention may be useful when the desired treatment involves areas or organs accessible by topical application, such as in the case of skin infections, wounds, or surgery. Accordingly, for topical administration, a pharmaceutical composition is preferably formulated to comprise a pyrazole carboxylic acid hydrazide compound described herein suspended or dissolved in a pharmaceutically acceptable carrier to provide a suitable ointment, cream, gel, jelly, or lotion suitable for topical application. An emollient may be present in such compositions to promote absorption of a compound described herein into or beneath the various layers of skin of an individual in need of treatment thereof. Pharmaceutically acceptable carriers for topical administration of a compound described herein may include, but are not limited to, mineral oil, liquid petroleum, white petroleum, propylene glycol, polyoxyethylene polyoxypropylene compound, emulsifying wax, sorbitan monostearate, polysorbate 60, cetyl esters wax, cetearyl alcohol, 2-octyldodecanol, benzyl alcohol, and water. Topical administration may also be accomplished via transdermal patches.

Pyrazole carboxylic acid hydrazide compounds may also be incorporated into cosmetic formulations to prevent or inhibit growth of bacteria and, thereby, prolong the shelf life of such formulations and/or provide antibacterial activity to the surface to which the cosmetic is applied.

The concentration of a particular compound in the formulations of the invention will vary depending upon a number of factors, including the dosage to be administered, and the route of administration. In general, the compounds of the invention can be provided in an aqueous physiological buffer solution containing about 0.1 to 10% w/v compound for parenteral administration. General dose ranges are from about 0.01 mg/kg to about 1 g/kg of body weight per day, for example, from about 0.01 mg/kg to 100 mg/kg of body weight per day. The dosage to be administered depends upon the type and extent of progression of the infection being addressed, the overall health of the patient, and the route of administration.

In one embodiment, a compound or composition of the invention is administered to an animal (for example, swine, chicken, or other commercially relevant livestock) or to a human patient that has been diagnosed with a bacterial infection. The compounds can also be administered to the animal or human to inhibit or reduce the likelihood of a bacterial infection, particularly in an animal or human susceptible to such an infection including, without limitation, a human patient who is immunodeficient or immunocompromised or a patient that has suffered a wound or has recently undergone a medical procedure.

In another embodiment, cultured eukaryotic cells or media used to suspend or grow such cells, may be incubated or otherwise contacted with a compound or composition described herein to inhibit or reduce the likelihood of bacterial infection.

### EXAMPLES

The following specific examples are to be construed as merely illustrative, and not limitative of the remainder of the disclosure in any way.

### Example 1

### Enzymatic Assays and Determination of Inhibitory Concentrations

Recombinant, purified pol IIIC and pol IIIE from Bacillus subtilis were obtained using known methods (Hammond et al., Gene 98: 29-36 (1991), regarding pol IIIC; Barnes et al., J. Bacteriol. 184, 3834-3838 (2002), regarding pol IIIE). Recombinant, purified pol IIIE from Escherichia coli was obtained from a commercial source (Enzyco, Denver, Colo.). The enzyme assays were performed in 96-well plate format using the same assay mix for all three enzymes. Each 25 μL assay contained 30 mM Tris, pH 7.5, 10 mM magnesium acetate, 4 mM dithiothreitol, 20% glycerol, with 10 μM dATP, dCTP, dGTP and dTTP (3H-labelled at 1.44 Ci/mmole) and 0.4 mg/mL activated calf thymus DNA as substrates. Dilutions of compounds were added to the plate in 1% DMSO (1 μL/well). Assays were initiated by the addition of 0.025 to 0.06 units of enzyme (1 unit is the amount required to incorporate 250 pmoles of [³H] dTMP in a standard assay), incubated for 10 min at 30° C. and terminated by the addition of 200 μL of cold 10% trichloroacetic acid, 10 mM sodium pyrophosphate. Precipitated labeled DNA was collected on glass fiber filter plates that had been pre-wet with ice cold 1 M HCl, 100 mM sodium pyrophosphate. The plates were washed with the HCl solution followed by ice cold 90% ethanol and then dried and counted in a liquid scintillation counter. The calculated IC₅₀ is the concentration needed to inhibit 50% of the DNA polymerase activity in the presence of all four dNTP substrates. In addition, percent inhibition values are calculated as the percent reduction in the activity of a DMSO (diluent) control. The percent inhibition values of representative compounds are listed in Table 3, below.

TABLE 3
Percent Enzymatic Inhibition

| Compound | B. subtilis | B. subtilis | E. coli pol |
|---|---|---|---|
| Number | pol IIIC | pol IIIE | IIIE |
| 1 | 14.3 | 52.2 | 0.4 |
| 2 | Inactive | 27.7 | 1.7 |
| 3 | 5.3 | 22.4 | Inactive |
| 4 | 15.8 | 22.9 | Inactive |
| 5 | 4.8 | 27.8 | 2.0 |
| 6 | 11.3 | 7.7 | 9.0 |
| 7 | Inactive | Inactive | Inactive |
| 8 | 19.7 | 33.4 | 59.6 |
| 9 | 29.5 | 21.7 | 55.5 |
| 10 | Inactive | 9.6 | Inactive |
| 11 | 1.3 | 16.7 | Inactive |
| 12 | Inactive | 7.6 | 7.2 |
| 13 | 2.9 | Inactive | 3.4 |
| 14 | 0.5 | 18.1 | Inactive |
| 15 | Inactive | 13.7 | Inactive |
| 16 | 9.9 | 21.8 | Inactive |
| 17 | 57.8 | 30.2 | Inactive |
| 18 | 26.6 | 32.6 | 58.8 |
| 19 | 27.3 | 10.1 | Inactive |
| 20 | 0.2 | 0.5 | Inactive |
| 21 | 64.6 | 36.8 | 41.5 |

Compounds were assayed at 80 μg/mL (190-260 μM) final concentration.

In the case of Compound 7, the test level of 80 μg/mL was not sufficient to show a significant inhibition of activity of any of the pol IIIC or pol IIIE species used in this study, however, as shown below, Compound 7 was able to inhibit growth of relevant test species of Gram-positive bacteria. Five of the compounds (Compounds 9, 17, 18, 19, and 21) showed greater than 20% inhibition of pol IIIC from B. subtilis. Eleven of the compounds (Compounds 1, 2, 3, 4, 5, 8, 9, 16, 17, 18 and 21) showed greater than 20% inhibition of pol IIIE from B. subtilis and four (Compounds 8, 9, 18 and 21) showed greater than 20% inhibition of E. coli pol IIIE as well. The data reveal that the pyrazole carboxylic acid hydrazide compounds described herein show some degree of specificity for Gram-positive bacterial DNA pol IIIE and may also inhibit Gram-positive bacterial DNA pol IIIC and Gram-negative DNA polymerases. Without wishing to be bound by theory, we believe that this activity profile is unique among all known antibiotics and may provide distinct advantages over such antibiotics (including, without limitation, the ability to treat or prevent bacterial infections that are resistant to other antibiotics).

### Example 2

### Determination of Inhibition of Bacterial Growth

Each compound was assayed against a panel of a strain of Bacillus subtilis, of strains of antibiotic-resistant and sensitive Enterococcus and Staphylococcus species, and of a strain of the Gram-negative bacterium Escherichia coli. Compounds were dissolved in DMSO at 100× the desired highest concentration and added to one well of a 96-well microassay plate. Two-fold serial drug dilutions were made across the plate in DMSO to yield 11 concentrations (usually 80-0.078 μg/mL) plus an untreated control. Drugs were then transferred to fresh plates using 1.5 μL per well. Log phase cultures were diluted to yield a concentration of 1×10⁵ colony forming units/mL in either LB or BHI medium and transferred to the plates containing drug for a final volume of 150 μL. Each well, including the DMSO controls, contained a final concentration of 1% DMSO. Plates were incubated with shaking at 37° C. for 16 to 18 hours. Cell growth was determined by measuring optical density (570 nm, 1 cm path length) in a microplate reader. MIC (minimum inhibitory concentration) is equivalent to the lowest concentration at which growth is not observed. The bacterial strains and MIC determinations have been well characterized and are described elsewhere (Tarantino et al., Antimicrob. Agents Chemother., 43: 1982-1987 (1999); Daly et al., Antimicrob. Agents Chemother., 44: 2217-2221 (2000)). The range of activity of compounds tested is about 0.625 μg/ml to greater than 80 μg/ml against the test strains of bacteria. Individual MIC values are listed in Table 4, below. Compound 17 showed the lowest MIC values, with compounds 1 and 18 rating second and third place, respectively. MIC values for the test strain of E. coli and several of the test strains of Gram-positive bacteria were not achieved for some of the compounds in this particular study, although such compounds clearly possess the critical property to inhibit a pol IIIC and/or pol IIIE activity, as shown above.

TABLE 4
Minimum Inhibitory Concentration (μg/mL)^{a} for Bacterial Growth

| Compound | | | | | | | | |
|---|---|---|---|---|---|---|---|---|
| Number | Bs | Sa | Sm | MRSA | Efcl | Efcm | VRE | Ec |
| 1 | 3 | 3 | 5 | 10 | 10 | 20 | 20 | >80 |
| 2 | 20 | 10 | 20 | 20 | 40 | 60 | 40 | >80 |
| 3 | >80 | 20 | 40 | 40 | 80 | 80 | 40 | >80 |
| 4 | 40 | 30 | 60 | 40 | >80 | 30 | >80 | >80 |
| 5 | 60 | 20 | 40 | 40 | 40 | 80 | 40 | >80 |
| 6 | >80 | 10 | >80 | 40 | 8 | 10 | 8 | >80 |
| 7 | 80 | 30 | 40 | 30 | >80 | >80 | >80 | >80 |
| 8 | >80 | >80 | >80 | >80 | >80 | >80 | >80 | >80 |
| 9 | >80 | >80 | >80 | >80 | >80 | >80 | >80 | >80 |
| 10 | >80 | >80 | >80 | >80 | >80 | >80 | >80 | >80 |
| 11 | >80 | >80 | >80 | >80 | >80 | >80 | >80 | >80 |
| 12 | >80 | >80 | >80 | >80 | >80 | >80 | >80 | >80 |
| 13 | >80 | >80 | >80 | >80 | >80 | >80 | >80 | >80 |
| 14 | >80 | 80 | 80 | 80 | >80 | >80 | 80 | >80 |
| 15 | >80 | 8 | >80 | >80 | >80 | 40 | >80 | >80 |
| 16 | >80 | >80 | >80 | >80 | >80 | >80 | 80 | >80 |
| 17 | 0.625 | 1.25 | 2.5 | 2.5 | 20 | 5 | 5 | >80 |
| 18 | 2.5 | 5 | 5 | 10 | 40 | 20 | 20 | >80 |
| 19 | 2.5 | 80 | >80 | 5 | >80 | >80 | 10 | >80 |
| 20 | >80 | >80 | >80 | 80 | >80 | >80 | 40 | >80 |
| 21 | >80 | >80 | >80 | >80 | >80 | >80 | >80 | >80 |

^{a}Bacterial strains were as follows: Bs, Bacillus subtilis strain BD54; Sa, Staphylococcus aureus strain IP 8; Sm, S. aureus Smith strain; MRSA, methicillin-resistant S. aureus strain 1094; Efcl, Enterococcus faecalis strain 29212; Efcm, E. faecium clinical isolate; VRE, vancomycin-resistant Enterococcus strain B42762; Ec, Escherichia coli strain J53.

### Example 3

### Mechanism of Inhibition Studies

DNA polymerase assays (for determining IC₅₀ values) were performed in the presence of all 4 dNTP substrates, or in the absence of one of the following; dCTP, dGTP or dATP with both pol IIIC and pol IIIE from B. subtilis, as shown in Tables 5 and 6, respectively, below.

TABLE 5
Substrate Competition in Gram-Positive Pol IIIC Inhibition

| | |
|---|---|
| Compound | B. subtilis Pol IIIC IC₅₀ (μM)^{a} |

| Number | 4 dNTPs | −dCTP | −dGTP | −dATP |
|---|---|---|---|---|
| 1 | 188 | 44 | 14 | 10 |
| 2 | Inactive | 287 | 406 | 99 |
| 3 | Inactive | 563 | 301 | 141 |
| 4 | Inactive | 228 | 98 | 81 |
| 5 | Inactive | 265 | 89 | 41 |
| 16 | Inactive | 96 | 60 | 89 |
| 17 | 36 | 2.6 | 1.8 | 3.1 |
| 18 | 912 | 6.5 | 5.5 | 23 |
| 19 | 23 | 3.3 | 5.1 | 9.0 |

^{a}IC₅₀ is the concentration needed to inhibit 50% of the DNA polymerase activity in the presence of all four dNTP substrates or in the absence of the indicated dNTP. A −dTTP assay was not performed since this was the dNTP that was labeled.

TABLE 6
Substrate Competition in Gram-Positive Pol IIIE Inhibition

| | |
|---|---|
| Compound | B. subtilis Pol IIIE IC₅₀ (μM)^{a} |

| No. | 4 dNTPs | −dCTP | −dGTP | −dATP |
|---|---|---|---|---|
| 1 | 166 | 62 | 51 | 75 |
| 2 | 604 | 104 | 78 | 57 |
| 3 | Inactive | 222 | 311 | 124 |
| 4 | 699 | 347 | 183 | 278 |
| 5 | 205 | 383 | 111 | 213 |
| 16 | Inactive | 156 | 318 | 117 |
| 17 | >780 | >780 | >780 | >780 |
| 18 | 278 | >1000 | >1000 | 345 |
| 19 | >841 | >841 | >841 | >841 |

^{a}IC₅₀ is the concentration needed to inhibit 50% of the DNA polymerase activity in the presence of all four dNTP substrates or in the absence of the indicated dNTP. A −dTTP assay was not performed since this was the dNTP that was labeled.

The IC₅₀ values for both Gram-positive DNA pol IIIC (Table 5) and DNA pol IIIE (Table 6) were relatively high in the presence of all four dNTPs (i.e., the lowest IC₅₀ was 23 μM). However, a dramatic lowering of IC₅₀ values was observed in the absence of any one dNTP (as much as a 166-fold reduction) for pol IIIC. Clearly, Compound 17, with an IC₅₀ of 1.8 μM in an assay where dGTP was eliminated, was the best inhibitor of this group. Interestingly, inhibition of pol IIIE was not improved by omitting one dNTP. By way of comparison, the anilinouracil Gram-positive pol IIIC inhibitors are specifically competitive with the substrate dGTP. The mechanism for the anilinouracil compounds is direct base pairing of the uracil moiety (mimicking guanine) with template cytosine and interaction of the anilino component with the aryl binding domain of the enzyme (Brown et al., Drugs Exp. Clin. Res., 12: 555-564 (1986)). The fact that the presence of all 4 dNTPs reduces the potency of the compounds of this invention and that omitting any one of the three dNTPs increases potency, suggests a new mechanism of action that is competitive with dNTP binding but not specific for a single dNTP, as are the anilinouracil class of compounds. Such a new mechanism of action would be particularly advantageous in addressing bacterial resistance that develops to the anilinouracil class of compounds.

Classical enzyme kinetic analyses were performed with Compound 17 and pol IIIC in which reaction velocity was measured as a function of each substrate (dGTP) concentration at several inhibitor concentrations (0, 20, and 60 μM). The results are graphed in a double reciprocal plot by the method of Lineweaver and Burk in FIG. 1A and show the effect of varying dGTP. The graph in FIG. 1A clearly shows that inhibition with Compound 17 is “uncompetitive” with dGTP. Similar results were obtained in assays in which dATP or dCTP were varied.

Compounds of the anilinouracil (AU) family, classical DNA polymerase inhibitors, inhibit pol IIIC strictly by “competitive” inhibition. An example is shown in the double reciprocal plot in FIG. 1B for the compound N3-hydroxybutyl 6-(3′-ethyl-4′-methylanilino) uracil (HB-EMAU), tested at 0, 1.6, and 4.8 μM. Such AU compounds compete with the dGTP substrate for binding to the enzyme (Clements et al., J. Biol. Chem. 250, 522-526 (1975)). “Noncompetive” inhibitors are exemplified by the class of HIV reverse transcriptase inhibitors, 4,5,6,7-tetrahydro-5-methylimidazo[4,5,1-jk][1,4]benzodiazepin-2(1H)-one (TIBO), i.e., TIBO compounds bind a site adjacent to the active site (Spence et al., Science 267, 988-993 (1995)). In contrast, the data in FIG. 1A clearly indicate that Compound 17, a pyrazole carboxylic acid hydrazide, inhibits pol IIIC by an “uncompetitive” mechanism. A classical uncompetitive inhibitor binds to the enzyme only after the substrate has bound (Devlin, T. M., ed., Textbook of Biochemistry With Clinical Correlations 5th ed. (Wiley-Liss, New York, 2002). As a result, both the apparent Vₘₐₓ and the apparent Kₘ decrease. Thus, the data indicate that pyrazole carboxylic acid hydrazides represent a novel class of pol IIIC inhibitors.

A number of embodiments of the invention have been described. Nevertheless, it will be understood that various modifications can be made without departing from the spirit and scope of the invention. Accordingly, other embodiments are within the scope of the following claims.

All publications, patent applications, patents, and other references mentioned herein are incorporated by reference in their entirety.

1. A method for treating an individual having or at risk of having a bacterial infection comprising:
administering to the individual a composition comprising a compound having the structure of formula 1a or 1b: 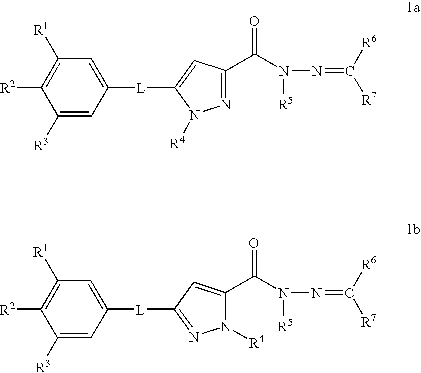
wherein R¹, R², and R³ are each, independently, selected from the group consisting of hydrogen (H), substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
R⁴ and R⁵ are, independently, selected from the group consisting of H, substituted or unsubstituted alkyl, substituted or unsubstituted alkenyl, substituted or unsubstituted alkynyl, and substituted or unsubstituted cycloalkyl; wherein when R⁴ is H, two tautomeric forms depicted by structures 1a and 1b may exist;
R⁶ and R⁷ are, independently, selected from the group consisting of H, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted alkylaryl, and substituted or unsubstituted heteroaryl, or R⁶ and R⁷ are atoms that form part of a aromatic or non-aromatic, heterocylic or carbocyclic ring or ring system, comprising either a monocyclic ring or a fused ring system, having ring atoms selected from the group consisting of substituted or unsubstituted carbon, nitrogen or sulfur; and
L may be absent or selected from the group consisting of linkers having 1-6 atoms in contiguous linear connectivity; or
an enantiomeric or diastereomeric form or a pharmaceutically acceptable salt thereof.

2. The method according to claim 1, wherein R¹, R² and R³ are each, independently, selected from the group consisting of hydrogen (H), substituted or unsubstituted C₁₋₆ alkyl, substituted or unsubstituted C₂₋₆ alkenyl, substituted or unsubstituted C₂₋₆ alkynyl, halo, amino, substituted or unsubstituted acyl, substituted or unsubstituted aryl, substituted or unsubstituted C₁₋₆ alkylamino, substituted or unsubstituted C₁₋₆ alkylaminoalkyl, substituted or unsubstituted C₁₋₆ alkoxy, substituted or unsubstituted C₁₋₆ alkoxyalkyl, substituted or unsubstituted C₁₋₆ alkylthio, substituted or unsubstituted C₁₋₆ alkylthioalkyl, nitro, hydroxyl, cyano, substituted or unsubstituted C₃₋₁₂ carbocyclyl, substituted or unsubstituted C₃₋₁₂ carbocyclylalkyl, substituted or unsubstituted C₃₋₁₂ carbocyclyloxy, substituted or unsubstituted C₃₋₁₂ carbocyclyloxyalkyl, substituted or unsubstituted C₃₋₁₂ carbocyclylamino, substituted or unsubstituted C₃₋₁₂ carbocyclylaminoalkyl, substituted or unsubstituted 3-12 membered heterocyclyl, substituted or unsubstituted 3-12 membered heterocyclyl-C₁-C₆ alkyl, substituted or unsubstituted 3-12 membered heterocyclyloxy, substituted or unsubstituted 3-12 membered heterocyclyloxy-C₁-C₆-alkyl, substituted or unsubstituted 3-12 membered heterocyclylamino, and substituted or unsubstituted 3-12 membered heterocyclylamino-C₁-C₆-alkyl;
R⁴ and R⁵ are, independently, selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl; wherein when R⁴ is H, two tautomeric forms depicted by structures 1a and 1b may exist;
R⁶ and R⁷ are, independently, selected from the group consisting of H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, and substituted or unsubstituted C₄₋₁₂ heteroaryl, or R⁶ and R⁷ are atoms that form part of a aromatic or non-aromatic, heterocylic or carbocyclic ring or ring system, comprising either a 3-6 membered monocyclic ring or a 6-12 membered fused ring system, having ring atoms selected from the group consisting of substituted or unsubstituted carbon, nitrogen, and sulfur; and
L may be absent or selected from the group consisting of linkers having 1-6 atoms in contiguous linear connectivity.

3. The method according to claim 2, wherein R⁴ and R⁵ are, independently, selected from the group consisting of H, substituted or unsubstituted C₁₋₆ alkyl, and substituted or unsubstituted C₃₋₈ cycloalkyl; wherein when R⁴ is H, two tautomeric forms depicted by structures 1a and 1b may exist;
R⁶ and R⁷ are, independently, selected from the group consisting of H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, and substituted or unsubstituted C₄₋₁₂ heteroaryl, or R⁶ and R⁷ are atoms that form part of a ring system having the structure: 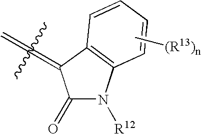
wherein R¹² is selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl,
each R¹³ is selected from the group consisting of substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
n is an integer selected from 0, 1, 2, 3, and 4; and
L may be absent or selected from the group consisting of linkers having 1-3 atoms in contiguous linear connectivity.

4. The method according to claim 3, wherein R⁴, R⁵, and R⁶ are H, and R⁷ is phenyl optionally substituted by one or more of C₁₋₆ alkyl, C₁₋₆ alkoxy, halo, amino, hydroxy, and halo, or R⁶ and R⁷ are atoms that form part of a ring system having the structure: 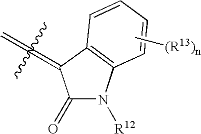
wherein R¹² is selected from the group consisting of H, substituted or unsubstituted C₁₋₆ alkyl, and substituted or unsubstituted C₃₋₈ cycloalkyl,
each R¹³ is selected from the group consisting of substituted or unsubstituted C₁₋₆ alkyl, substituted or unsubstituted C₂₋₆ alkenyl, substituted or unsubstituted C₂₋₆ alkynyl, halo, amino, substituted or unsubstituted C₁₋₆ acyl, substituted or unsubstituted aryl, substituted or unsubstituted C₁₋₆ alkoxy, substituted or unsubstituted C₁₋₆ alkoxyalkyl, nitro, hydroxyl, and cyano,
n is an integer selected from 0, 1, and 2; and
L is absent.

5. The method according to claim 1, wherein the compound has the structure of formula 2: 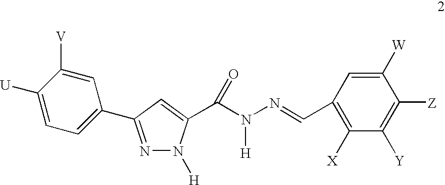
wherein U, V, W, X, Y and Z may be the same or different and are, independently, selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
or an enantiomeric or diastereomeric form or a pharmaceutically acceptable salt thereof.

6. The method according to claim 5, wherein U, V, W, X, Y, and Z are, independently, selected from the group consisting of H, C₁₋₄ alkyl, C₃₋₆ cycloalkyl, C₁₋₄ alkoxy, halo, hydroxyl, and amino.

7. The method according to claim 5, wherein the compound is selected from the group consisting of Compounds 1-19, having the structure indicated by formula 2 and the table below:

| | |
|---|---|
| | 2 |
| 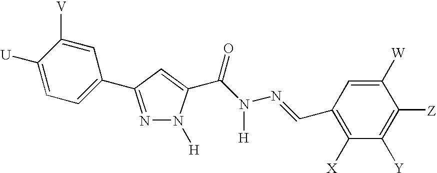 | |
| Compound | | | | | | |
|---|---|---|---|---|---|---|
| Number | U | V | W | X | Y | Z |
| 1 | H | H | OCH₃ | H | OCH₃ | OCH₃ |
| 2 | H | OCH₃ | H | H | OCH₃ | OCH₃ |
| 3 | H | OCH₃ | H | H | OCH₂CH₃ | OH |
| 4 | H | H | H | OH | H | OH |
| 5 | H | H | H | H | OCH₃ | OH |
| 6 | H | H | Br | OH | H | H |
| 7 | H | OCH₂CH₃ | H | OH | OCH₃ | H |
| 8 | CH₃ | H | H | OH | OCH₃ | H |
| 9 | H | H | H | OH | H | H |
| 10 | H | OCH₃ | H | H | OH | H |
| 11 | H | H | H | H | H | CH₃ |
| 12 | H | H | C₄H₉ | H | C₄H₉ | OH |
| 13 | OCH₃ | H | H | H | H | H |
| 14 | H | OCH₃ | H | H | H | OH |
| 15 | Cl | H | H | H | OCH₃ | OCH₃ |
| 16 | OCH₃ | H | H | H | H | OH |
| 17 | OCH₃ | H | OCH₃ | H | OCH₃ | OCH₃ |
| 18 | OCH₃ | H | H | H | OCH₃ | OCH₃ |
| 19 | OC₂H₅ | H | H | H | OCH₃ | OH |

8. The method according to claim 1, wherein the compound has the structure of formula 3: 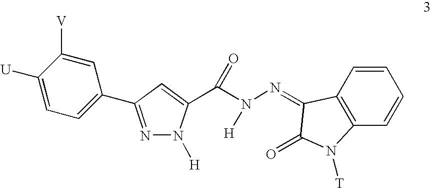
wherein, U and V may be the same or different and are, independently, selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl; and
T is selected from the group consisting of H, substituted or unsubstituted alkyl, substituted or unsubstituted alkenyl, substituted or unsubstituted alkynyl, and substituted or unsubstituted cycloalkyl.

9. The method according to claim 8, wherein U and V are independently selected from the group consisting of H, C₁₋₄ alkyl, C₁₋₄ alkoxy, and aryl, and T is selected from the group consisting of H and C₁₋₄ alkyl.

10. The method according to claim 8, wherein the compound is selected from the group consisting of Compounds 20 and 21, having the structure indicated by formula 3 and the table below:

| | |
|---|---|
| | 3 |
| 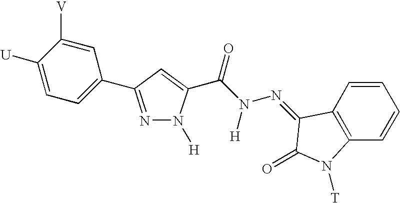 | |
| Compound Number | T | U | V |
|---|---|---|---|
| 20 | C₂H₅ | H | H |
| 21 | C₂H₅ | H | C₆H₅ |

11. The method according to claim 1, wherein the composition further comprises a pharmaceutically acceptable carrier.

12. The method according to claim 11, wherein the composition further comprises an additional ingredient selected from the group consisting of another antibiotic, an antiviral compound, an anti-cancer compound, a vitamin, a trace metal supplement, an ion, and combinations thereof.

13. The method according to claim 1, wherein the composition is administered to the individual parenterally or non-parenterally.

14. The method according to claim 1, wherein the composition is administered to the individual through a route selected from the group consisting of intravenously, subcutaneously, intramuscularly, intraorbitally, ophthalmically, intraventricularly, intracranially, intracapsularly, intraspinally, intracisternally, intraperitoneally, intranasally, orally, buccally, rectally, vaginally, topically, and combinations thereof.

15. The method according to claim 1, wherein the composition is administered to the individual by aerosol, by scarification, or by surgical implant.

16. The method according to claim 1, wherein the bacterial infection is caused by Gram-positive bacteria or mycoplasma bacteria.

17. The method according to claim 16, wherein the bacteria are Gram-positive bacteria.

18. The method according to claim 17, wherein the Gram-positive bacteria are selected from the group consisting of Streptococcus, Enterococcus, Staphylococcus, Bacillus, Clostridium, Listeria, and combinations thereof.

19. The method according to claim 1, wherein the bacterial infection is caused by Gram-negative bacteria.

20. The method according to claim 19, wherein the Gram-negative bacteria are selected from the group consisting of Escherichia, Salmonella, Pseudomonas, Helicobacter, Legionella, Shigella, Yersinia, Neisseria, and combinations thereof.

21. A method of inhibiting growth of bacteria comprising:
contacting the bacteria with a compound having a structure of formula 1a or 1b: 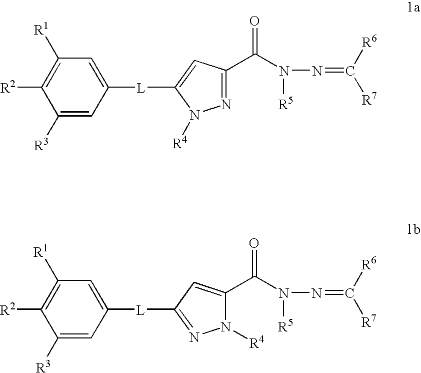
wherein R¹, R², and R³ are each, independently, selected from the group consisting of hydrogen (H), substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
R⁴ and R⁵ are, independently, selected from the group consisting of H, substituted or unsubstituted alkyl, substituted or unsubstituted alkenyl, substituted or unsubstituted alkynyl, and substituted or unsubstituted cycloalkyl; wherein when R⁴ is H, two tautomeric forms depicted by structures 1a and 1b may exist;
R⁶ and R⁷ are, independently, selected from the group consisting of H, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted alkylaryl, and substituted or unsubstituted heteroaryl, or R⁶ and R⁷ are atoms that form part of a aromatic or non-aromatic, heterocylic or carbocyclic ring or ring system, comprising either a monocyclic ring or a fused ring system, having ring atoms selected from the group consisting of substituted or unsubstituted carbon, nitrogen, and sulfur; and
L may be absent or selected from the group consisting of linkers having 1-6 atoms in contiguous linear connectivity; or
an enantiomeric or diastereomeric form or a pharmaceutically acceptable salt thereof.

22. The method according to claim 21, wherein R¹, R², and R³ are each, independently, selected from the group consisting of hydrogen (H), substituted or unsubstituted C₁₋₆ alkyl, substituted or unsubstituted C₂₋₆ alkenyl, substituted or unsubstituted C₂₋₆ alkynyl, halo, amino, substituted or unsubstituted acyl, substituted or unsubstituted aryl, substituted or unsubstituted C₁₋₆ alkylamino, substituted or unsubstituted C₁₋₆ alkylaminoalkyl, substituted or unsubstituted C₁₋₆ alkoxy, substituted or unsubstituted C₁₋₆ alkoxyalkyl, substituted or unsubstituted C₁₋₆ alkylthio, substituted or unsubstituted C₁₋₆ alkylthioalkyl, nitro, hydroxyl, cyano, substituted or unsubstituted C₃₋₁₂ carbocyclyl, substituted or unsubstituted C₃₋₁₂ carbocyclylalkyl, substituted or unsubstituted C₃₋₁₂ carbocyclyloxy, substituted or unsubstituted C₃₋₁₂ carbocyclyloxyalkyl, substituted or unsubstituted C₃₋₁₂ carbocyclylamino, substituted or unsubstituted C₃₋₁₂ carbocyclylaminoalkyl, substituted or unsubstituted 3-12 membered heterocyclyl, substituted or unsubstituted 3-12 membered heterocyclyl-C₁-C₆ alkyl, substituted or unsubstituted 3-12 membered heterocyclyloxy, substituted or unsubstituted 3-12 membered heterocyclyloxy-C₁-C₆-alkyl, substituted or unsubstituted 3-12 membered heterocyclylamino, and substituted or unsubstituted 3-12 membered heterocyclylamino-C₁-C₆-alkyl;
R⁴ and R⁵ are, independently, selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl; wherein when R⁴ is H, two tautomeric forms depicted by structures 1a and 1b may exist;
R⁶ and R⁷ are, independently, selected from the group consisting of H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, and substituted or unsubstituted C₄₋₁₂ heteroaryl, or R⁶ and R⁷ are atoms that form part of a aromatic or non-aromatic, heterocylic or carbocyclic ring or ring system, comprising either a 3-6 membered monocyclic ring or a 6-12 membered fused ring system, having ring atoms selected from the group consisting of substituted or unsubstituted carbon, nitrogen, and sulfur; and
L may be absent or selected from the group consisting of linkers having 1-6 atoms in contiguous linear connectivity.

23. The method according to claim 22, wherein R⁴ and R⁵ are, independently, selected from the group consisting of H, substituted or unsubstituted C₁₋₆ alkyl, and substituted or unsubstituted C₃₋₈ cycloalkyl; wherein when R⁴ is H, two tautomeric forms depicted by structures 1a and 1b may exist;
R⁶ and R⁷ are, independently, selected from the group consisting of H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, and substituted or unsubstituted C₄₋₁₂ heteroaryl, or R⁶ and R⁷ are atoms that form part of a ring system having the structure: 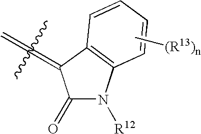
wherein R¹² is selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl,
each R¹³ is selected from the group consisting of substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
n is an integer selected from 0, 1, 2, 3, and 4; and
L may be absent or selected from the group consisting of linkers having 1-3 atoms in contiguous linear connectivity.

24. The method according to claim 23, wherein R⁴, R⁵, and R⁶ are H, and R⁷ is phenyl optionally substituted by one or more of C₁₋₆ alkyl, C₁₋₆ alkoxy, halo, amino, hydroxy, and halo, or R⁶ and R⁷ are atoms that form part of a ring system having the structure: 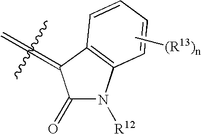
wherein R¹² is selected from the group consisting of H, substituted or unsubstituted C₁₋₆ alkyl, and substituted or unsubstituted C₃₋₈ cycloalkyl,
each R¹³ is selected from the group consisting of substituted or unsubstituted C₁₋₆ alkyl, substituted or unsubstituted C₂₋₆ alkenyl, substituted or unsubstituted C₂₋₆ alkynyl, halo, amino, substituted or unsubstituted C₁₋₆ acyl, substituted or unsubstituted aryl, substituted or unsubstituted C₁₋₆ alkoxy, substituted or unsubstituted C₁₋₆ alkoxyalkyl, nitro, hydroxyl, and cyano,
n is an integer selected from 0, 1, and 2; and
L is absent.

25. The method according to claim 21, wherein the compound has the structure of formula 2: 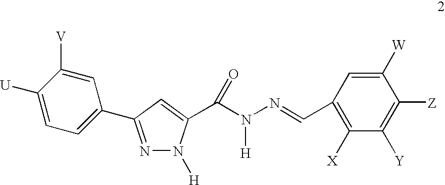
wherein U, V, W, X, Y, and Z may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl; or an enantiomeric or diastereomeric form or a pharmaceutically acceptable salt thereof.

26. The method according to claim 25, wherein U, V, W, X, Y, and Z are independently selected from the group consisting of H, C₁₋₄ alkyl, C₃₋₆ cycloalkyl, C₁₋₄ alkoxy, halo, hydroxyl, and amino.

27. The method according to claim 25, wherein the compound is selected from the group consisting of Compounds 1-19, having the structure indicated by formula 2 and the table below:

| | |
|---|---|
| | 2 |
| 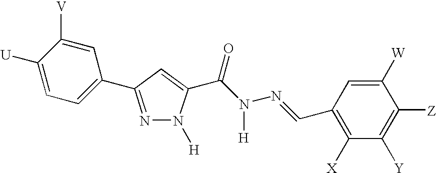 | |
| Compound | | | | | | |
|---|---|---|---|---|---|---|
| Number | U | V | W | X | Y | Z |
| 1 | H | H | OCH₃ | H | OCH₃ | OCH₃ |
| 2 | H | OCH₃ | H | H | OCH₃ | OCH₃ |
| 3 | H | OCH₃ | H | H | OCH₂CH₃ | OH |
| 4 | H | H | H | OH | H | OH |
| 5 | H | H | H | H | OCH₃ | OH |
| 6 | H | H | Br | OH | H | H |
| 7 | H | OCH₂CH₃ | H | OH | OCH₃ | H |
| 8 | CH₃ | H | H | OH | OCH₃ | H |
| 9 | H | H | H | OH | H | H |
| 10 | H | OCH₃ | H | H | OH | H |
| 11 | H | H | H | H | H | CH₃ |
| 12 | H | H | C₄H₉ | H | C₄H₉ | OH |
| 13 | OCH₃ | H | H | H | H | H |
| 14 | H | OCH₃ | H | H | H | OH |
| 15 | Cl | H | H | H | OCH₃ | OCH₃ |
| 16 | OCH₃ | H | H | H | H | OH |
| 17 | OCH₃ | H | OCH₃ | H | OCH₃ | OCH₃ |
| 18 | OCH₃ | H | H | H | OCH₃ | OCH₃ |
| 19 | OC₂H₅ | H | H | H | OCH₃ | OH |

28. The method according to claim 21, wherein the compound has the structure of formula 3: 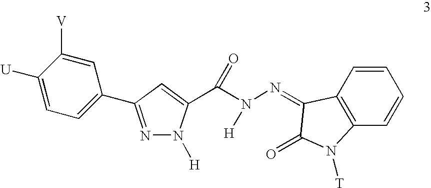
wherein, U and V may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl; and
T is selected from the group consisting of H, substituted or unsubstituted alkyl, substituted or unsubstituted alkenyl, substituted or unsubstituted alkynyl, and substituted or unsubstituted cycloalkyl.

29. The method according to claim 28, wherein U and V are independently selected from the group consisting of H, C₁₋₄ alkyl, C₁₋₄ alkoxy, and aryl, and T is selected from the group consisting of H and C₁₋₄ alkyl.

30. The method according to claim 28, wherein the compound is selected from the group consisting of Compounds 20 and 21, having the structure indicated by formula 3 and the table below:

| | |
|---|---|
| | 3 |
| 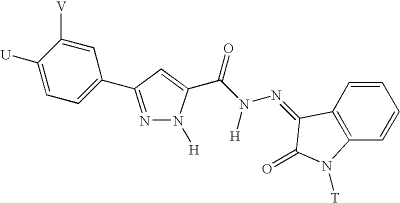 | |
| Compound Number | T | U | V |
|---|---|---|---|
| 20 | C₂H₅ | H | H |
| 21 | C₂H₅ | H | C₆H₅ |

31. The method according to claim 21, wherein the bacteria are Gram-positive bacteria or mycoplasma bacteria.

32. The method according to claim 31, wherein the bacteria are Gram-positive bacteria.

33. The method according to claim 32, wherein the Gram-positive bacteria are selected from the group consisting of Streptococcus, Enterococcus, Staphylococcus, Bacillus, Clostridium, Listeria, and combinations thereof.

34. The method according to claim 21, wherein the bacteria are Gram-negative bacteria.

35. The method according to claim 34, wherein the Gram-negative bacteria are selected from the group consisting of Escherichia, Salmonella, Pseudomonas, Helicobacter, Legionella, Shigella, Yersinia, Neisseria, and combinations thereof.

36. A method of inhibiting the activity of a DNA polymerase III comprising:
contacting the DNA polymerase III with a compound having a structure of formula 1a or 1b: 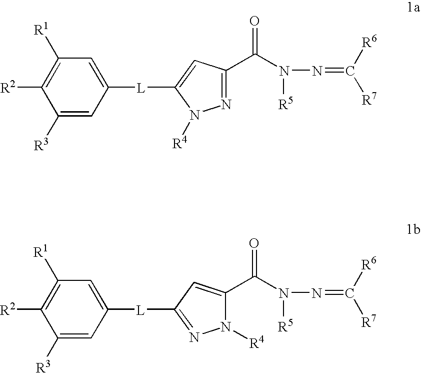
wherein R¹, R², and R³ are each, independently, selected from the group consisting of hydrogen (H), substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
R⁴ and R⁵ are, independently, selected from the group consisting of H, substituted or unsubstituted alkyl, substituted or unsubstituted alkenyl, substituted or unsubstituted alkynyl, and substituted or unsubstituted cycloalkyl; wherein when R⁴ is H, two tautomeric forms depicted by structures 1a and 1b may exist;
R⁶ and R⁷ are, independently, selected from the group consisting of H, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted alkylaryl, and substituted or unsubstituted heteroaryl, or R⁶ and R⁷ are atoms that form part of a aromatic or non-aromatic, heterocylic or carbocyclic ring or ring system, comprising either a monocyclic ring or a fused ring system, having ring atoms selected from the group consisting of substituted or unsubstituted carbon, nitrogen, and sulfur; and
L may be absent or selected from the group consisting of linkers having 1-6 atoms in contiguous linear connectivity; or
an enantiomeric or diastereomeric form or a pharmaceutically acceptable salt thereof.

37. The method according to claim 36, wherein R¹, R², and R³ are each, independently, selected from the group consisting of hydrogen (H), substituted or unsubstituted C₁₋₆ alkyl, substituted or unsubstituted C₂₋₆ alkenyl, substituted or unsubstituted C₂₋₆ alkynyl, halo, amino, substituted or unsubstituted acyl, substituted or unsubstituted aryl, substituted or unsubstituted C₁₋₆ alkylamino, substituted or unsubstituted C₁₋₆ alkylaminoalkyl, substituted or unsubstituted C₁₋₆ alkoxy, substituted or unsubstituted C₁₋₆ alkoxyalkyl, substituted or unsubstituted C₁₋₆ alkylthio, substituted or unsubstituted C₁₋₆ alkylthioalkyl, nitro, hydroxyl, cyano, substituted or unsubstituted C₃₋₁₂ carbocyclyl, substituted or unsubstituted C₃₋₁₂ carbocyclylalkyl, substituted or unsubstituted C₃₋₁₂ carbocyclyloxy, substituted or unsubstituted C₃₋₁₂ carbocyclyloxyalkyl, substituted or unsubstituted C₃₋₁₂ carbocyclylamino, substituted or unsubstituted C₃₋₁₂ carbocyclylaminoalkyl, substituted or unsubstituted 3-12 membered heterocyclyl, substituted or unsubstituted 3-12 membered heterocyclyl-C₁-C₆ alkyl, substituted or unsubstituted 3-12 membered heterocyclyloxy, substituted or unsubstituted 3-12 membered heterocyclyloxy-C₁-C₆-alkyl, substituted or unsubstituted 3-12 membered heterocyclylamino, and substituted or unsubstituted 3-12 membered heterocyclylamino-C₁-C₆-alkyl;
R⁴ and R⁵ are, independently, selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl; wherein when R⁴ is H, two tautomeric forms depicted by structures 1a and 1b may exist;
R⁶ and R⁷ are, independently, selected from the group consisting of H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, and substituted or unsubstituted C₄₋₁₂ heteroaryl, or R⁶ and R⁷ are atoms that form part of a aromatic or non-aromatic, heterocylic or carbocyclic ring or ring system, comprising either a 3-6 membered monocyclic ring or a 6-12 membered fused ring system, having ring atoms selected from the group consisting of substituted or unsubstituted carbon, nitrogen, and sulfur; and
L may be absent or selected from the group consisting of linkers having 1-6 atoms in contiguous linear connectivity.

38. The method according to claim 37, wherein R⁴ and R⁵ are, independently, selected from the group consisting of H, substituted or unsubstituted C₁₋₆ alkyl, and substituted or unsubstituted C₃₋₈ cycloalkyl; wherein when R⁴ is H, two tautomeric forms depicted by structures 1a and 1b may exist;
R⁶ and R⁷ are, independently, selected from the group consisting of H, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₂ cycloalkyl, substituted or unsubstituted aryl, substituted or unsubstituted arylalkyl, substituted or unsubstituted C₁₋₆ alkylaryl, and substituted or unsubstituted C₄₋₁₂ heteroaryl, or R⁶ and R⁷ are atoms that form part of a ring system having the structure: 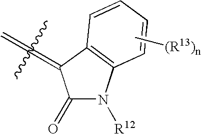
wherein R¹² is selected from the group consisting of H, substituted or unsubstituted C₁₋₆ alkyl, substituted or unsubstituted C₂₋₆ alkenyl, substituted or unsubstituted C₂₋₆ alkynyl, and substituted or unsubstituted C₃₋₈ cycloalkyl,
each R¹³ is selected from the group consisting of substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
n is an integer selected from 0, 1, 2, 3, and 4; and
L may be absent or selected from the group consisting of linkers having 1-3 atoms in contiguous linear connectivity.

39. The method according to claim 38, wherein R⁴, R⁵, and R⁶ are H, and R⁷ is phenyl optionally substituted by one or more of C₁₋₆ alkyl, C₁₋₆ alkoxy, halo, amino, hydroxy, and halo, or R⁶ and R⁷ are atoms that form part of a ring system having the structure: 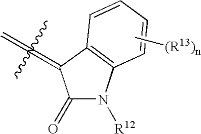
wherein R¹² is selected from the group consisting of H, substituted or unsubstituted C₁₋₆ alkyl, and substituted or unsubstituted C₃₋₈ cycloalkyl,
each R¹³ is selected from the group consisting of substituted or unsubstituted C₁₋₆ alkyl, substituted or unsubstituted C₂₋₆ alkenyl, substituted or unsubstituted C₂₋₆ alkynyl, halo, amino, substituted or unsubstituted C₁₋₆ acyl, substituted or unsubstituted aryl, substituted or unsubstituted C₁₋₆ alkoxy, substituted or unsubstituted C₁₋₆ alkoxyalkyl, nitro, hydroxyl, and cyano,
n is an integer selected from 0, 1, and 2; and
L is absent.

40. The method according to claim 36, wherein the compound has the structure of formula 2: 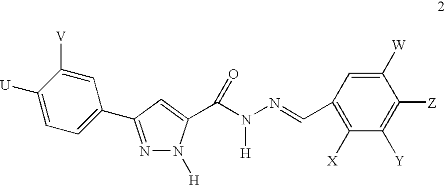
wherein U, V, W, X, Y, and Z may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
or an enantiomeric or diastereomeric form or a pharmaceutically acceptable salt thereof.

41. The method according to claim 40, wherein U, V, W, X, Y, and Z are independently selected from the group consisting of H, C₁₋₄ alkyl, C₃₋₆ cycloalkyl, C₁₋₄ alkoxy, halo, hydroxyl, and amino.

42. The method according to claim 40, wherein the compound is selected from the group consisting of Compounds 1-19, having the structure indicated by formula 2 and the table below:

| | |
|---|---|
| | 2 |
| 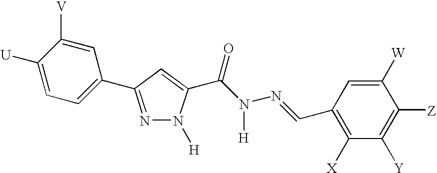 | |
| Compound | | | | | | |
|---|---|---|---|---|---|---|
| Number | U | V | W | X | Y | Z |
| 1 | H | H | OCH₃ | H | OCH₃ | OCH₃ |
| 2 | H | OCH₃ | H | H | OCH₃ | OCH₃ |
| 3 | H | OCH₃ | H | H | OCH₂CH₃ | OH |
| 4 | H | H | H | OH | H | OH |
| 5 | H | H | H | H | OCH₃ | OH |
| 6 | H | H | Br | OH | H | H |
| 7 | H | OCH₂CH₃ | H | OH | OCH₃ | H |
| 8 | CH₃ | H | H | OH | OCH₃ | H |
| 9 | H | H | H | OH | H | H |
| 10 | H | OCH₃ | H | H | OH | H |
| 11 | H | H | H | H | H | CH₃ |
| 12 | H | H | C₄H₉ | H | C₄H₉ | OH |
| 13 | OCH₃ | H | H | H | H | H |
| 14 | H | OCH₃ | H | H | H | OH |
| 15 | Cl | H | H | H | OCH₃ | OCH₃ |
| 16 | OCH₃ | H | H | H | H | OH |
| 17 | OCH₃ | H | OCH₃ | H | OCH₃ | OCH₃ |
| 18 | OCH₃ | H | H | H | OCH₃ | OCH₃ |
| 19 | OC₂H₅ | H | H | H | OCH₃ | OH |

43. The method according to claim 36, wherein the compound has the structure of formula 3: 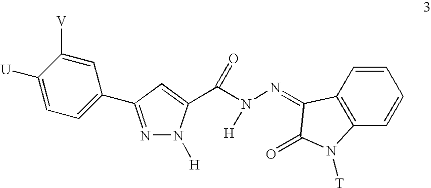
wherein, U and V may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl; and
T is selected from the group consisting of H, substituted or unsubstituted alkyl, substituted or unsubstituted alkenyl, substituted or unsubstituted alkynyl, and substituted or unsubstituted cycloalkyl.

44. The method according to claim 43, wherein U and V are independently selected from the group consisting of H, C₁₋₄ alkyl, C₁₋₄ alkoxy, and aryl, and T is selected from the group consisting of H and C₁₋₄ alkyl.

45. The method according to claim 43, wherein the compound is selected from the group consisting of Compounds 20 and 21, having the structure indicated by formula 3 and the table below:

| | |
|---|---|
| | 3 |
| 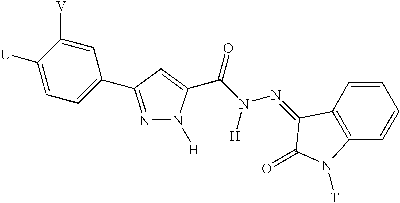 | |
| Compound Number | T | U | V |
|---|---|---|---|
| 20 | C₂H₅ | H | H |
| 21 | C₂H₅ | H | C₆H₅ |

46. The method according to claim 36, wherein the DNA polymerase III is a pol IIIC class or a pol IIIE class enzyme.

47. The method according to claim 36, wherein the DNA polymerase III is from Gram-positive bacteria or mycoplasma bacteria.

48. The method according to claim 47, wherein the bacterial DNA polymerase III is from Gram-positive bacteria.

49. The method according to claim 48, wherein the Gram-positive bacteria are selected from the group consisting of Streptococcus, Enterococcus, Staphylococcus, Bacillus, Clostridium, Listeria, and combinations thereof.

50. The method according to claim 46, wherein the DNA polymerase III is a pol IIIE class enzyme.

51. The method according to claim 50, wherein the pol IIIE class enzyme is from Gram-negative bacteria.

52. The method according to claim 51, wherein the Gram-negative bacteria are selected from the group consisting of Escherichia, Salmonella, Pseudomonas, Helicobacter, Legionella, Shigella, Yersinia, Neisseria, and combinations thereof.

53. (canceled)

54. (canceled)

55. A pharmaceutical composition for the treatment or prevention of a bacterial infection comprising an amount of a pyrazole carboxylic acid hydrazide effective to inhibit bacterial growth and a pharmaceutically acceptable carrier, wherein said pyrazole carboxylic acid hydrazide compound has the structure of formula 1a or 1b: 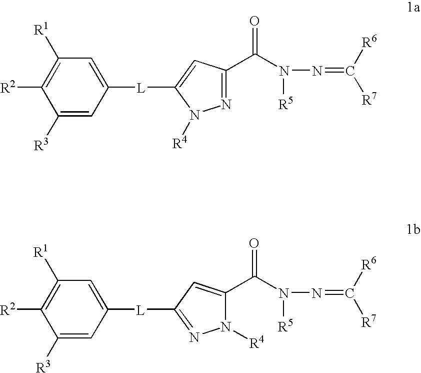
wherein R¹, R², and R³ are each, independently, selected from the group consisting of hydrogen (H), substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
R⁴ and R⁵ are, independently selected from the group consisting of H, substituted or unsubstituted alkyl, substituted or unsubstituted alkenyl, substituted or unsubstituted alkynyl, and substituted or unsubstituted cycloalkyl; wherein when R⁴ is H, two tautomeric forms depicted by structures 1a and 1b may exist:
R⁶ and R⁷ are, independently, selected from the group consisting of H, substituted or unsubstituted alkyl, substituted or unsubstituted alkenyl, substituted or unsubstituted alkynyl, substituted or unsubstituted cycloalkyl, substituted or unsubstituted aryl, substituted or unsubstituted arylalkyl, substituted or unsubstituted alkylaryl, and substituted or unsubstituted heteroaryl, or R⁶ and R⁷ are atoms that form part of a aromatic or non-aromatic, heterocylic or carbocyclic ring or ring system, comprising either a monocyclic ring or a fused ring system, having ring atoms selected from the group consisting of substituted or unsubstituted carbon, nitrogen or sulfur; and
L may be absent or selected from the group consisting of linkers having 1-6 atoms in contiguous linear connectivity; or
an enantiomeric or diastereomeric form or a pharmaceutically acceptable salt thereof.

56. The pharmaceutical composition according to claim 55, wherein said pyrazole carboxylic acid hydrazide compound has the structure of formula 2: 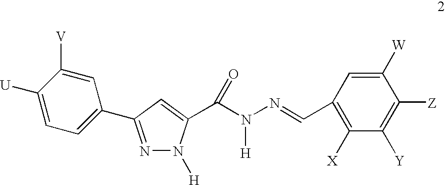
wherein U, V, W, X, Y, and Z may be the same or different and are independently selected from the group consisting of H, substituted or unsubstituted alkyl, substituted or unsubstituted alkenyl, substituted or unsubstituted alkynyl, halo, amino, substituted or unsubstituted acyl, substituted or unsubstituted aryl, substituted or unsubstituted alkylamino, substituted or unsubstituted alkylaminoalkyl, substituted or unsubstituted alkoxy, substituted or unsubstituted alkoxyalkyl, substituted or unsubstituted alkylthio, substituted or unsubstituted alkylthioalkyl, nitro, hydroxyl, cyano, substituted or unsubstituted carbocyclyl, substituted or unsubstituted carbocyclylalkyl, substituted or unsubstituted carbocyclyloxy, substituted or unsubstituted carbocyclyloxyalkyl, substituted or unsubstituted carbocyclylamino, substituted or unsubstituted carbocyclylaminoalkyl, substituted or unsubstituted heterocyclyl, substituted or unsubstituted heterocyclylalkyl, substituted or unsubstituted membered heterocyclyloxy, substituted or unsubstituted heterocyclyloxyalkyl, substituted or unsubstituted heterocyclylamino, and substituted or unsubstituted heterocyclylaminoalkyl;
or an enantiomeric or diastereomeric form or a pharmaceutically acceptable salt thereof.
